SEQUENCE LISTING (34 sequences)

SEQ ID NO: 1 - DNA, 20 bp, Artificial Sequence
  synthetic construct; primer
gaattcccagtaagtgcggg

SEQ ID NO: 2 - DNA, 20 bp, Artificial Sequence
  synthetic construct; primer
gggcagggacttaatcaacg

SEQ ID NO: 3 - DNA, 21 bp, Artificial Sequence
  synthetic construct; primer
acaacttcttcagcccttccc

SEQ ID NO: 4 - DNA, 20 bp, Artificial Sequence
  synthetic construct; primer
tctgcaccacctctccttgg

SEQ ID NO: 5 - DNA, 20 bp, Artificial Sequence
  synthetic construct; primer
agcaaacagcctggcatagc

SEQ ID NO: 6 - DNA, 20 bp, Artificial Sequence
  synthetic construct; primer
acttgcagcccatctgctct

SEQ ID NO: 7 - DNA, 21 bp, Artificial Sequence
  synthetic construct; primer
gaactctttcggtcgtacccc

SEQ ID NO: 8 - DNA, 19 bp, Artificial Sequence
  synthetic construct; primer
cgtgaacgttgcccatcat

SEQ ID NO: 9 - DNA, 19 bp, Artificial Sequence
  synthetic construct; primer
cttgagaagagggccgcac

SEQ ID NO: 10 - DNA, 20 bp, Artificial Sequence
  synthetic construct; primer
aacgttgcccatcatcatca

SEQ ID NO: 11 - DNA, 20 bp, Artificial Sequence
  synthetic construct; primer
gagacagcagggctcactcc

SEQ ID NO: 12 - DNA, 20 bp, Artificial Sequence
  synthetic construct; primer
tggcccttcttggtcttcag

SEQ ID NO: 13 - DNA, 20 bp, Artificial Sequence
  synthetic construct; primer
attggcagattgccctggat

SEQ ID NO: 14 - DNA, 20 bp, Artificial Sequence
  synthetic construct; primer
gccttcggagcagaacatca

SEQ ID NO: 15 - DNA, 20 bp, Artificial Sequence
  synthetic construct; primer
tgacctggataagcacctgg

SEQ ID NO: 16 - DNA, 20 bp, Artificial Sequence
  synthetic construct; primer
aagctctgaagctcctgctg

SEQ ID NO: 17 - DNA, 20 bp, Artificial Sequence
  synthetic construct; primer
atggcagcagctccagactt

SEQ ID NO: 18 - DNA, 20 bp, Artificial Sequence
  synthetic construct; primer
cccaacctccaaaccatcat

SEQ ID NO: 19 - DNA, 20 bp, Artificial Sequence
  synthetic construct; primer
gcctcatcccgttccttgtt

SEQ ID NO: 20 - DNA, 20 bp, Artificial Sequence
  synthetic construct; primer
cctgggcatcgctgtcttta

SEQ ID NO: 21 - DNA, 20 bp, Artificial Sequence
  synthetic construct; primer
cgacctggagtgtatgagcc

SEQ ID NO: 22 - DNA, 20 bp, Artificial Sequence
  synthetic construct; primer
acacacttgttttcgtgccg

SEQ ID NO: 23 - DNA, 20 bp, Artificial Sequence
  synthetic construct; primer
gatgtaagtcgaggcccaag

SEQ ID NO: 24 - DNA, 20 bp, Artificial Sequence
  synthetic construct; primer
gcaggcattgcaaaggtagt

SEQ ID NO: 25 - DNA, 21 bp, Artificial Sequence
  synthetic construct; primer
actccgcaaggtggtagatga

SEQ ID NO: 26 - DNA, 21 bp, Artificial Sequence
  synthetic construct; primer
tccacttccacagtcaatcca

SEQ ID NO: 27 - DNA, 21 bp, Artificial Sequence
  synthetic construct; primer
agcggcaagagtgagatttct

SEQ ID NO: 28 - DNA, 21 bp, Artificial Sequence
  synthetic construct; primer
cctccaggttattctccaggg

SEQ ID NO: 29 - DNA, 20 bp, Artificial Sequence
  synthetic construct; primer
gagatcgccacctacaggaa

SEQ ID NO: 30 - DNA, 20 bp, Artificial Sequence
  synthetic construct; primer
tcctccgtagccagaagaga

SEQ ID NO: 31 - DNA, 19 bp, Artificial Sequence
  synthetic construct; primer
cggctgcgagagaaattgc

SEQ ID NO: 32 - DNA, 22 bp, Artificial Sequence
  synthetic construct; primer
ccactttccgttcaaggtcaag

SEQ ID NO: 33 - DNA, 24 bp, Artificial Sequence
  synthetic construct; primer
cggttccgatgccctgaggctctt

SEQ ID NO: 34 - DNA, 24 bp, Artificial Sequence
  synthetic construct; primer
cgtcacacttcatgatggaattga

(19) United States
(12) Patent Application Publication
(10) Pub. No.: US 2018/0346989 A1
(43) Pub. Date: Dec. 6, 2018

(54) METHODS OF DIAGNOSING AND TREATING BREAST CANCER

(71) Applicant: BOARD OF REGENTS, THE UNIVERSITY OF TEXAS SYSTEM, Austin, TX (US)

(72) Inventors: Rong Li, San Antonio, TX (US); Xiaowen Zhang, San Antonio, TX (US); Huai-Chin Chiang, San Antonio, TX (US)

(21) Appl. No.: 15/571,819

(22) PCT Filed: May 4, 2016
(86) PCT No.: PCT/US2016/030730

§ 371 Date: Nov. 3, 2017

Related U.S. Application Data
(60) Provisional application No. 62/156,686, filed on May 4, 2015.

(51) Int. Cl. C12Q 1/6886; G01N 33/574; C12N 15/113

(52) U.S. Cl. CPC C12Q 1/6886; G01N 33/57415; C12N 2310/14; C12Q 2600/156; C12Q 2600/118; C12N 15/113

(57) ABSTRACT

Disclosed are methods of diagnosing whether a subject is at risk of developing breast cancer comprising measuring the level of R-loop in a biological sample. Also disclosed are methods of treating breast cancer in a subject comprising administering to said subject a therapeutically effective amount of a given therapeutic when the subject is diagnosed with increased risk of developing breast cancer by the steps that include measuring the level of R-loop in a biological sample. The measured level of R-loop in a biological sample can be compared to a control sample from a non-BRCA mutation carrier. Disclosed are methods of treating a subject having an increase in breast epithelium R-loop comprising administering a treatment to the subject that reduces or eliminates R-loop, wherein the subject is a BRCA1 mutation carrier.

### CROSS-REFERENCE TO RELATED APPLICATIONS

This application claims benefit of U.S. Provisional Application No. 62/156,686, filed May 4, 2015 and is hereby incorporated herein by reference in its entirety.

### REFERENCE TO SEQUENCE LISTING

The Sequence Listing submitted May 4, 2016 as a text file named “21105_0027P1 Sequence Listing.txt,” created on May 3, 2016, and having a size of 7,123 bytes is hereby incorporated by reference pursuant to 37 C.F.R. § 1.52(e)(5).

### BACKGROUND

Women carrying germ-line mutations of BRCA1 and BRCA2 have significantly increased risk of developing breast and ovarian cancers. Both BRCA1 and BRCA2 play roles in reducing R-loops, a DNA-RNA hybrid structure and by-product of transcription. It is well known that individuals with one mutated germ-line BRCA allele are at an increased risk for developing breast cancer. However, there is no available method to pre-screen individuals in the general population who may harbor deleterious BRCA mutations and/or have elevated risk of developing breast cancer. The use of the association of BRCA1/2 mutation carriers and elevated R-loop signals could be a useful tool for diagnosing individuals with a risk of developing breast cancer.

### BRIEF SUMMARY

Disclosed are methods of diagnosing whether a subject is at risk of developing breast cancer, comprising a) obtaining a biological sample from the subject; b) measuring the level of R-loop in the biological sample; c) conducting at least one hybridization assay of the biological sample so as to obtain physical data to determine whether the level of R-loop in the biological sample is higher than the level of R-loop in a control sample from a non-BRCA mutation carrier; d) comparing the level of R-loop in the biological sample with the level of R-loop in a control sample from a non-BRCA mutation carrier; and e) identifying the subject is at risk of developing breast cancer if the physical data indicate that the level of R-loop in the biological sample is higher than the level of R-loop in a control sample from a non-BRCA mutation carrier.

Disclosed are methods of diagnosing whether a subject is at risk of developing breast cancer, comprising a) obtaining a biological sample from the subject; b) measuring the level of R-loop in the biological sample; c) conducting at least one hybridization assay of the biological sample so as to obtain physical data to determine whether the level of R-loop in the biological sample is higher than the level of R-loop in a control sample from a non-BRCA mutation carrier; d) comparing the level of R-loop in the biological sample with the level of R-loop in a control sample from a non-BRCA mutation carrier; and e) identifying the subject is at risk of developing breast cancer if the physical data indicate that the level of R-loop in the biological sample is higher than the level of R-loop in a control sample from a non-BRCA mutation carrier, wherein the subject is a BRCA mutation carrier. In some instances, the BRCA mutation carrier is a BRCA1 or BRCA2 mutation carrier.

Disclosed are methods of diagnosing whether a subject is at risk of developing breast cancer, comprising a) obtaining a biological sample from the subject; b) measuring the level of R-loop in the biological sample; c) conducting at least one hybridization assay of the biological sample so as to obtain physical data to determine whether the level of R-loop in the biological sample is higher than the level of R-loop in a control sample from a non-BRCA mutation carrier; d) comparing the level of R-loop in the biological sample with the level of R-loop in a control sample from a non-BRCA mutation carrier; and e) identifying the subject is at risk of developing breast cancer if the physical data indicate that the level of R-loop in the biological sample is higher than the level of R-loop in a control sample from a non-BRCA mutation carrier, wherein the non-BRCA mutation carrier is a subject having two wild type copies of the BRCA gene.

Disclosed are methods of diagnosing whether a subject is at risk of developing breast cancer, comprising a) obtaining a biological sample from the subject; b) measuring the level of R-loop in the biological sample; c) conducting at least one hybridization assay of the biological sample so as to obtain physical data to determine whether the level of R-loop in the biological sample is higher than the level of R-loop in a control sample from a non-BRCA mutation carrier; d) comparing the level of R-loop in the biological sample with the level of R-loop in a control sample from a non-BRCA mutation carrier; and e) identifying the subject is at risk of developing breast cancer if the physical data indicate that the level of R-loop in the biological sample is higher than the level of R-loop in a control sample from a non-BRCA mutation carrier, wherein the hybridization assay includes an ELISPOT assay, ELISA, fluorescent immunoassays, two-antibody sandwich assays, a flow-through or strip test format, PCR, Real time PCR, Reverse Transcription-PCR (RT-PCR), immunohistochemistry, or DNA/RNA immunoprecipitation.

Disclosed are methods of diagnosing whether a subject is at risk of developing breast cancer, comprising a) obtaining a biological sample from the subject; b) measuring the level of R-loop in the biological sample; c) conducting at least one hybridization assay of the biological sample so as to obtain physical data to determine whether the level of R-loop in the biological sample is higher than the level of R-loop in a control sample from a non-BRCA mutation carrier; d) comparing the level of R-loop in the biological sample with the level of R-loop in a control sample from a non-BRCA mutation carrier; and e) identifying the subject is at risk of developing breast cancer if the physical data indicate that the level of R-loop in the biological sample is higher than the level of R-loop in a control sample from a non-BRCA mutation carrier, the hybridization assay is carried out with an R-loop antibody.

Disclosed are methods of diagnosing whether a subject is at risk of developing breast cancer, comprising a) obtaining a biological sample from the subject; b) measuring the level of R-loop in the biological sample; c) conducting at least one hybridization assay of the biological sample so as to obtain physical data to determine whether the level of R-loop in the biological sample is higher than the level of R-loop in a control sample from a non-BRCA mutation carrier; d) comparing the level of R-loop in the biological sample with the level of R-loop in a control sample from a non-BRCA mutation carrier; and e) identifying the subject is at risk of developing breast cancer if the physical data indicate that the level of R-loop in the biological sample is higher than the level of R-loop in a control sample from a non-BRCA mutation carrier, wherein the sample comprises one or more of tissue, blood, bone marrow, plasma, serum, urine, and feces. In some instances, the sample comprises breast tissue. In some instances, the sample comprises epithelial cells. The epithelial cells can be luminal epithelial cells.

Disclosed are methods of diagnosing whether a subject is at risk of developing breast cancer, comprising a) obtaining a biological sample from the subject; b) measuring the level of R-loop in the biological sample; c) conducting at least one hybridization assay of the biological sample so as to obtain physical data to determine whether the level of R-loop in the biological sample is higher than the level of R-loop in a control sample from a non-BRCA mutation carrier; d) comparing the level of R-loop in the biological sample with the level of R-loop in a control sample from a non-BRCA mutation carrier; and e) identifying the subject is at risk of developing breast cancer if the physical data indicate that the level of R-loop in the biological sample is higher than the level of R-loop in a control sample from a non-BRCA mutation carrier, further comprising administering to said subject a therapeutically effective amount of a given therapeutic.

Disclosed are methods for treating breast cancer in a subject, comprising administering to said subject a therapeutically effective amount of a given therapeutic when the subject is diagnosed with increased risk of developing breast cancer by the steps of a) obtaining a biological sample from the subject; b) measuring the level of R-loop in the biological sample; c) conducting at least one hybridization assay of the biological sample so as to obtain physical data to determine whether the level of R-loop in the biological sample is higher than the level of R-loop in a control sample from a non-BRCA mutation carrier; d) comparing the level of R-loop in the biological sample with the level of R-loop in a control sample from a non-BRCA mutation carrier; and e) identifying the subject is at risk of developing breast cancer if the physical data indicate that the level of R-loop in the biological sample is higher than the level of R-loop in a control sample from a non-BRCA mutation carrier.

Disclosed are methods of treating a subject having an increase in breast epithelium R-loop comprising administering a treatment to the subject that reduces or eliminates R-loop, wherein the subject is a BRCA1 mutation carrier.

Disclosed are methods of treating a subject having an increase in breast epithelium R-loop comprising administering a treatment to the subject that reduces or eliminates R-loop, wherein the subject is a BRCA1 mutation carrier, wherein the treatment is increasing expression and/or activity of RNase H, which degrades the RNA component in the R-loop structure, or decreasing COBRA1 expression and/or activity.

Disclosed are methods of reducing tumor incidence in BRCA1-deficient subjects comprising administering a treatment that reduces or eliminates Cobra1 activity.

Disclosed are methods of reducing tumor incidence in BRCA1-deficient subjects comprising administering a treatment that reduces or eliminates Cobra1 activity, wherein the treatment comprises siRNA that targets Cobra1 mRNA.

Disclosed are methods of increasing mammary gland development in Cobra-deficient subjects comprising administering a treatment that reduces or eliminates BRCA1 activity.

Disclosed are methods of increasing mammary gland development in Cobra-deficient subjects comprising administering a treatment that reduces or eliminates BRCA1 activity, wherein the treatment comprises siRNA. The siRNA can target BRCA1 mRNA.

Disclosed are methods of increasing mammary gland development in Cobra-deficient subjects comprising administering a treatment that alters transcription of puberty-related genes, estrogen-responsive genes or progesterone-responsive genes.

Disclosed are methods of increasing mammary gland development in Cobra-deficient subjects comprising administering a treatment that alters transcription of one or more puberty-related genes, estrogen-responsive genes or progesterone-responsive genes, wherein the puberty-related genes are Gata3, Prlr, Ramp2, Vwf, Prom2, Acot1, or a combination thereof. Other puberty-related genes include, but are not limited to, those genes listed in FIG. 19.

Disclosed are methods of increasing mammary gland development in Cobra-deficient subjects comprising administering a treatment that alters transcription of one or more puberty-related genes, estrogen-responsive genes or progesterone-responsive genes, wherein the estrogen-responsive genes are 2410081M15RIK, 6430706D22RIK, ACOT1, ACTB, ARL4A, BCL6B, CTSH, CXCL9, EMCN, GBP6, GGTA1, HOXA7, PABPC1, PDLIM1, PDLIM2, PROM2, PTPN14, SLCO2B1, STARD10, TMEM2, WIPI1, or a combination thereof.

Disclosed are methods of increasing mammary gland development in Cobra-deficient subjects comprising administering a treatment that alters transcription of one or more puberty-related genes, estrogen-responsive genes or progesterone-responsive genes, wherein the progesterone-responsive genes are 5730593F17RIK, CCDC80, CDH13, CLDN5, CRYZ, EDN1, IRX1, NOXO1, PKP2, PRKCDBP, PSEN2, SLC7A3, SPNB3, or a combination thereof.

Disclosed are methods of increasing mammary gland development in Cobra-deficient subjects comprising administering a treatment that alters transcription of puberty-related genes, estrogen-responsive genes or progesterone-responsive genes, wherein a treatment that alters transcription comprises a treatment that increases transcription.

Disclosed are methods of increasing mammary gland development in Cobra-deficient subjects comprising administering a treatment that alters transcription of puberty-related genes, estrogen-responsive genes or progesterone-responsive genes, wherein a treatment that alters transcription comprises a treatment that decreases transcription.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B, and 1C show that DKO rescues ductal developmental defect in CKO. (a) Whole mounts of mammary glands from 8-wk virgin mice. The boundary of the ductal area is highlighted. Scale bars: 1 mm. Images are representatives of at least 6 animals. (b) Measurement of the average ductal length at 4 developmental time points. The numbers of animals used for each of the four time points (6, 8, 12, 24 weeks) are: WT (4, 7, 7, 12 mice), BKO (3, 3, 4, 4 mice), CKO (3, 6, 5, 8 mice), and DKO (4, 7, 4, 4 mice). (c) Flow cytometry analysis of various mammary gland compartments from 16-wk virgin mice. Stromal cells: CD49f-EpCAM-, luminal epithelial cells: CD49fmedEpCAMhigh, myoepithelial cells: CD49fhighEpCAMmed. The numbers of animals used are: WT (4), BKO (3), CKO (3), and DKO (4). *P<0.05, **P<0.01 by Student's t-test. Statistical analysis was conducted between CKO and WT, and between DKO and CKO. Error bars represent standard error of the mean (s.e.m.).

FIGS. 2A, 2B, 2C, and 2D show that DKO rescues alveolar and lactogenic defects associated with CKO and BKO. (a-b) Whole mounts of mammary glands one day postpartum. Scale bar: 1 mm in (a), and 500 μm in (b). (c) Hematoxylin and eosin (H&E) stain of the lobular-alveolar structure in mammary glands of mice one day postpartum. Scale bar: 100 μm. (d) Immunohistochemistry for total milk proteins in mammary glands of mice one day postpartum. Scale bar: 50 μm. Images in this figure are representatives of at least 4 animals in each genotype.

FIGS. 3A, 3B, and 3C show aberrant pubertal gene expression in CKO is partially rescued in DKO. (a) Heatmap illustrates the gene expression changes in mammary epithelial cells of CKO and DKO as compared to their corresponding WT littermates (n=3) at three time points (4, 6, 8 weeks). The gene expression levels in WT are set as 1. (b) Expression patterns for two representative pubertal genes that are affected by CKO and partially rescued by DKO. The lowest expression level in each graph is set at 1. (c) Confirmation of the microarray data by gene-specific RT-PCR for a number of pubertal genes. The result is average of values from 3 animals in each genotype. Error bars represent s.e.m. *P<0.05. Statistical analysis was conducted between WT and CKO, and between CKO and DKO.

FIGS. 4A, 4B, and 4C show that Cobra1 deletion reduces mammary tumor incidence and abundance of luminal progenitor cells in BKO. (a) Curve for tumor incidence. *P<0.05. The number of animals used in each group is indicated. Log-rank test was used to estimate the statistical significance. (b) Enumeration of mature luminal (CD49fmedEpCAMhighCD49b−) and progenitor cells (CD49fmedEpCAMhighCD49b+). The numbers of animals used are: WT (4), BKO (3), CKO (3), and DKO (4). **P<0.01. (c) H&E stain of mammary ducts from WT and KO animals. Scale bar: 50 μm.

FIGS. 5A, 5B, 5C, 5D, and 5E show that Cobra1 deletion does not rescue DSB repair deficiency in DKO. (a) Diagram of the GFP reporter assay for measuring HR efficiency. I-Sce1: restriction enzyme. iGFP: internal GFP fragment as the template for HR. (b) Top: Immunoblot of COBRA1 and BRCA1 for assessing siRNA knockdown efficiency with control (Con) oligos or ones targeting human BRCA1 (-B) and COBRA1 (-C) in HeLa cells. Bottom: Percentage of GFP+ cells as a result of HR-mediated DSB repair. Results are average of three independent experiments. *P<0.05 by Student's t-test. (c) Mice of 8-wk old were pulse-labeled with BrdU, irradiated (20 Gy), and mammary glands were harvested 3 hr later for immunostaining for γH2AX and BrdU. Scale bar: 5 μm. (d) The same samples as shown in (c) were stained for Rad51 and BrdU. Scale bar: 5 μm. (e) Percentage of Rad51+/BrdU+ mammary epithelial cells. *P<0.05. Error bars represent s.e.m. The numbers of animals used are indicated below the graph.

FIGS. 6A, 6B, 6C, and 6D show that COBRA1 contributes to R loop accumulation in BKO. (a) Immunofluorescence staining for R-loop structure in mammary ducts of 8-wk virgin mice. Scale bars: 20 μm (left) and 5 μm (right). (b) Quantitation of relative R-loop intensity in 8-week old animals. The numbers of animals used in each group are: WT (9), BKO (9), CKO (5), and DKO (8). *P<0.05. (c) Average reads per million for RNAPII ChIP-seq, NELF-A ChIP-seq, NELF-B/COBRA1 ChIP-seq, and DRIP-seq surrounding the TSS regions in mammary epithelial cells. Reads from two biological repeats were merged for RNAPII ChIP-seq and DRIP-seq. (d) Venn diagram indicating the overlapping genes with TSS-enriched signals for RNAPII, NELF-A/B, and R loops.

FIGS. 7A, 7B, and 7C show elevated R-loop signal in normal breast tissue from BRCA1 mutation carriers. (a) Low and high magnification images of R-loop staining in samples from non-carriers and BRCA1 mutation carriers, with and without pre-treatment of RNase H. Scale bar: 20 μm (left) and 5 μm (right). (b) Low and high magnification images of R-loop staining in BRCA1 mutation carriers, showing different staining signals in the luminal epithelial compartment and basal/stromal compartments. Scale bar: 20 μm (top) and 5 μm (bottom). (c) Quantitation of the R-loop intensity in luminal epithelial and basal/stromal compartments in the non-carrier group (n=12), BRCA1 mutation carrier group (n=12), and BRCA1 mutation carrier pre-treated with RNase H group (n=5). *P<0.05. **P<0.01.

FIG. 8 shows a model that illustrates the functional antagonism between BRCA1 and COBRA1 during normal mammary gland development and tumorigenesis.

FIGS. 9A and 9B show the depletion of COBRA1 and BRCA1 in mammary epithelial cells of virgin KO mice. (a) IHC of COBRA1 in 8-week virgin mice. Representative result from at least 5 sets of animals. Scale bar: 50 □m. (b) RT-PCR analysis of COBRA1 and BRCA1 mRNA levels from sorted luminal mammary epithelial cells. Representative result from more than 6 sets of WT and mutant animals. Also shown are relative expression levels of COBRA1 and BRCA1 in myoepithelial and luminal epithelial cells of WT mammary glands. 18S rRNA was used for normalization. Note that COBRA1 is expressed to similar levels in both epithelial compartments, whereas BRCA1 is predominantly expressed in the luminal compartment. The relatively high residual levels of COBRA1 mRNA in sorted luminal epithelial samples of CKO mice could be due to minor contamination with myoepithelial cells.

FIG. 10 shows homozygous Cobra1 deletion results in ductal growth defects. Whole mounts of 8-wk virgin mice. Representative images from at least 4 animals in each genotype. Scale bar: 1 mm.

FIGS. 11A, 11B, 11C, and 11D show the sorting of luminal and myoepithelial cells. (a) Representative flow cytometry results indicating the typical gating for debris exclusion, doublet discrimination, selection of lineage-negative/live cells, and separation of luminal, myoepithelial cells and stromal cells. (b) Cell surface markers and fluorochromes used in the flow cytometry. (c) Validation of cell sorting efficiency by RT-PCR of known luminal (K18) and myoepithelial cell (K5 and K14) markers. 18S rRNA was used as the normalization control. (d) Enumeration of ERα+ luminal cells by IHC in WT and KO mammary glands. The numbers of animals used are: WT (10), BKO (8), CKO (10), and DKO (10).

FIG. 12 shows lack of signs of morphogenic rescue in 6-week DKO. Whole mounts of mammary gland tissue from 6-week animals. The images are representatives of at least 3 animals in each genotype. Scale bar: 1 mm.

FIGS. 13A and 13B show the developmental defect in CKO cannot be rescued by Ink4-Arf deletion. (a) Whole mounts of 8-wk virgin mice. Representative images from at least 3 mice in each genotype group. Scale bar: 1 mm. (b) mRNA analysis for COBRA1, p16INK4a, and p19ARF by RT-PCR, using sorted luminal mammary epithelial cells. Error bars represent standard deviation.

FIGS. 14A and 14B who the developmental defect in CKO cannot be rescued by Trp53 deletion. (a) Whole mounts of 8-wk virgin mice. Representative images from at least 3 mice in each genotype group. Scale bar: 1 mm. (b) mRNA analysis for Cobra1 and Trp53, using sorted luminal mammary epithelial cells. Error bars represent standard deviation.

FIG. 15 shows immunofluorescence staining with luminal epithelial and myoepithelial markers K8 and K14, respectively. Scale bar: 50 μm.

FIG. 16 shows examples of genes with TSS-enriched signals for RNAPII, NELF, and R-loops in mammary epithelium.

FIGS. 17A, 17B, and 17C show elevated R-loop signals in BRCA1 mutation carriers. (a) Four examples of non-carriers and BRCA1 mutation carriers stained for R-loop and DAPI from the Lombardi Cancer Center. Each image is from a specific donor. Scale bar: 20 μm. (b) Higher magnification images of R-loop staining in non-carriers and BRCA1 mutation carriers from the same individuals as shown in A. Scale bar: 5 μm. (c) Quantitation of the R-loop intensity in the non-carrier group (n=12) and BRCA1 mutation carrier group (n=13). **P<0.01.

FIG. 18 shows a relapse-free survival curve for TNBC patients with low or high COBRA1 expression.

FIG. 19 shows a table of 6 wks CKO affected and pubertal related gene list.

FIGS. 20A, B, and C show R-loops in BRCA1 mutant luminal cells preferentially accumulate at luminal super-enhancers. Average normalized reads for R loops at (A) TSS, (B) super-enhancers, and (C).

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. If a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a therapeutic” includes a plurality of such therapeutics, reference to “the subject” is a reference to one or more subjects and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

As used herein, by “administering” is meant a method of giving a dosage of a composition, such as a therapeutic, to a subject in need thereof. The compositions described herein can be administered by any acceptable route known in the art and including, e.g., parenteral, dermal, transdermal, ocular, inhalation, buccal, sublingual, perilingual, nasal, rectal, topical, and oral administration. Parenteral administration includes intra-arterial, intravenous, intraperitoneal, subcutaneous, and intramuscular administration. The preferred method of administration can vary depending on various factors (e.g., the components of the composition being administered, the condition being treated and its severity, and the age, weight, and health of the patient).

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” breast cancer can refer to reducing the symptoms of breast cancer, reducing the spread of breast cancer and/or eliminating breast cancer. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of an inventive vaccine nanocarrier to a subject.

Ranges may be expressed herein as from “about” one particular value, and/or to “about” another particular value. When such a range is expressed, also specifically contemplated and considered disclosed is the range¬ from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Those skilled in the art will recognize, or be able to ascertain using no more than routine experimentation, many equivalents to the specific embodiments of the method and compositions described herein. Such equivalents are intended to be encompassed by the following claims.

### B. Methods of Diagnosing Risk of Developing Breast Cancer

Disclosed are methods of diagnosing whether a subject is at risk of developing breast cancer, comprising a) obtaining a biological sample from the subject; b) measuring the level of R-loop in the biological sample by conducting at least one hybridization assay of the biological sample so as to obtain physical data to determine whether the level of R-loop in the biological sample is higher than the level of R-loop in a control sample from a non-BRCA mutation carrier; c) comparing the level of R-loop in the biological sample with the level of R-loop in a control sample from a non-BRCA mutation carrier; and d) identifying the subject is at risk of developing breast cancer if the physical data indicate that the level of R-loop in the biological sample is higher than the level of R-loop in a control sample from a non-BRCA mutation carrier.

The subject can be a BRCA mutation carrier. The BRCA mutation can be in BRCA1 or BRCA2. Therefore, the subject can be a BRCA1 or BRCA2 mutation carrier. A non-BRCA mutation carrier can be a subject having two wild type copies of the BRCA gene.

Disclosed are methods of diagnosing whether a subject is at risk of developing breast cancer, comprising a) obtaining a biological sample from the subject; b) measuring the level of R-loop in the biological sample by conducting at least one hybridization assay of the biological sample so as to obtain physical data to determine whether the level of R-loop in the biological sample is higher than the level of R-loop in a control sample from a non-BRCA mutation carrier; c) comparing the level of R-loop in the biological sample with the level of R-loop in a control sample from a non-BRCA mutation carrier; and d) identifying the subject is at risk of developing breast cancer if the physical data indicate that the level of R-loop in the biological sample is higher than the level of R-loop in a control sample from a non-BRCA mutation carrier, wherein the hybridization assay includes an ELISPOT assay, ELISA, fluorescent immunoassays, two-antibody sandwich assays, a flow-through or strip test format, PCR, Real time PCR , Reverse Transcription-PCR (RT-PCR), immunohistochemistry, or DNA/RNA immunoprecipitation.

Disclosed are methods of diagnosing whether a subject is at risk of developing breast cancer, comprising a) obtaining a biological sample from the subject; b) measuring the level of R-loop in the biological sample by conducting at least one hybridization assay of the biological sample so as to obtain physical data to determine whether the level of R-loop in the biological sample is higher than the level of R-loop in a control sample from a non-BRCA mutation carrier; c) comparing the level of R-loop in the biological sample with the level of R-loop in a control sample from a non-BRCA mutation carrier; and d) identifying the subject is at risk of developing breast cancer if the physical data indicate that the level of R-loop in the biological sample is higher than the level of R-loop in a control sample from a non-BRCA mutation carrier, wherein the hybridization assay is carried out with an R-loop antibody.

Disclosed are methods of diagnosing whether a subject is at risk of developing breast cancer, comprising a) obtaining a biological sample from the subject; b) measuring the level of R-loop in the biological sample by conducting at least one hybridization assay of the biological sample so as to obtain physical data to determine whether the level of R-loop in the biological sample is higher than the level of R-loop in a control sample from a non-BRCA mutation carrier; c) comparing the level of R-loop in the biological sample with the level of R-loop in a control sample from a non-BRCA mutation carrier; and d) identifying the subject is at risk of developing breast cancer if the physical data indicate that the level of R-loop in the biological sample is higher than the level of R-loop in a control sample from a non-BRCA mutation carrier, wherein the sample comprises one or more of tissue, blood, bone marrow, plasma, serum, urine, and feces. In some instances, the sample can comprise breast tissue or epithelial cells. For example, epithelial cells can be luminal epithelial cells.

Disclosed are methods of diagnosing whether a subject is at risk of developing breast cancer, comprising a) obtaining a biological sample from the subject; b) measuring the level of R-loop in the biological sample by conducting at least one hybridization assay of the biological sample so as to obtain physical data to determine whether the level of R-loop in the biological sample is higher than the level of R-loop in a control sample from a non-BRCA mutation carrier; c) comparing the level of R-loop in the biological sample with the level of R-loop in a control sample from a non-BRCA mutation carrier; and d) identifying the subject is at risk of developing breast cancer if the physical data indicate that the level of R-loop in the biological sample is higher than the level of R-loop in a control sample from a non-BRCA mutation carrier, further comprising administering to said subject a therapeutically effective amount of a given therapeutic. A therapeutic can be any known breast cancer therapeutics such as, but not limited to, chemotherapy, radiation, targeted therapies, such as Her-2 specific therapies, or hormone therapy.

### C. Methods for Treating Breast Cancer

Disclosed are methods for treating breast cancer in a subject, comprising administering to said subject a therapeutically effective amount of a given therapeutic when the subject is diagnosed with increased risk of developing breast cancer by the steps of (a) obtaining a biological sample from the subject; (b) measuring the level of R-loop in the biological sample by conducting at least one hybridization assay of the biological sample so as to obtain physical data to determine whether the level of R-loop in the biological sample is higher than the level of R-loop in a control sample from a non-BRCA mutation carrier; (c) comparing the level of R-loop in the biological sample with the level of R-loop in a control sample from a non-BRCA mutation carrier; and (d) identifying the subject is at risk of developing breast cancer if the physical data indicate that the level of R-loop in the biological sample is higher than the level of R-loop in a control sample from a non-BRCA mutation carrier.

The subject can be a BRCA mutation carrier. The BRCA mutation can be in BRCA1 or BRCA2. Therefore, the subject can be a BRCA1 or BRCA2 mutation carrier. A non-BRCA mutation carrier can be a subject having two wild type copies of the BRCA gene.

Disclosed are methods for treating breast cancer in a subject, comprising administering to said subject a therapeutically effective amount of a given therapeutic when the subject is diagnosed with increased risk of developing breast cancer by the steps of (a) obtaining a biological sample from the subject; (b) measuring the level of R-loop in the biological sample by conducting at least one hybridization assay of the biological sample so as to obtain physical data to determine whether the level of R-loop in the biological sample is higher than the level of R-loop in a control sample from a non-BRCA mutation carrier; (c) comparing the level of R-loop in the biological sample with the level of R-loop in a control sample from a non-BRCA mutation carrier; and (d) identifying the subject is at risk of developing breast cancer if the physical data indicate that the level of R-loop in the biological sample is higher than the level of R-loop in a control sample from a non-BRCA mutation carrier, wherein the hybridization assay includes an ELISPOT assay, ELISA, fluorescent immunoassays, two-antibody sandwich assays, a flow-through or strip test format, PCR, Real time PCR, Reverse Transcription-PCR (RT-PCR), immunohistochemistry, or DNA/RNA immunoprecipitation. In some instances, the hybridization assay can be carried out with an R-loop antibody.

The sample can comprise one or more of tissue, blood, bone marrow, plasma, serum, urine, and feces. In some instances, the sample can comprise breast tissue or epithelial cells. For example, epithelial cells can be luminal epithelial cells.

A therapeutic can be any known breast cancer therapeutics such as, but not limited to, chemotherapy, radiation, targeted therapies, such as Her-2 specific therapies, or hormone therapy.

D. Methods of Treating Subjects with Increased R-Loops

Disclosed are methods of treating a subject having an increase in breast epithelium R-loop comprising administering a treatment to the subject that reduces or eliminates R-loop, wherein the subject is a BRCA1 mutation carrier.

Disclosed are methods of treating a subject having an increase in breast epithelium R-loop comprising administering a treatment to the subject that reduces or eliminates R-loop, wherein the subject is a BRCA1 mutation carrier, wherein the treatment can be increasing expression and/or activity of RNase H, which degrades the RNA component in the R-loop structure; decreasing COBRA1 expression and/or activity; or a combination thereof.

### E. Methods of Reducing Tumors

Disclosed are methods of reducing tumor incidence in BRCA1-deficient subjects comprising administering a treatment that reduces or eliminates Cobra1 activity.

Disclosed are methods of reducing tumor incidence in BRCA1-deficient subjects comprising administering a treatment that reduces or eliminates Cobra1 activity, wherein the treatment comprises siRNA that targets Cobra1 mRNA. COBRA1 (aka NELF-B) is an integral subunit of the four-subunit complex and depletion of any one subunit can abolish the NELF activity. Therefore, targeting the other NELF subunits, NELF-A, -C/D, and -E, can also be used for COBRA1 inactivation/elimination.

### F. Methods of Increasing Mammary Gland Development

Disclosed are methods of increasing mammary gland development in Cobra-deficient subjects comprising administering a treatment that reduces or eliminates BRCA1 activity.

Disclosed are methods of increasing mammary gland development in Cobra-deficient subjects comprising administering a treatment that reduces or eliminates BRCA1 activity, wherein the treatment comprises siRNA.

Disclosed are methods of increasing mammary gland development in Cobra-deficient subjects comprising administering a treatment that reduces or eliminates BRCA1 activity, wherein the treatment comprises siRNA, wherein the siRNA targets BRCA1 mRNA. BRCA1 can form a stable dimeric complex with BARD1 and the protein stability of these two proteins can be mutually dependent. Therefore, depletion of BARD1 can also be used to reduce or eliminate BRCA1 activity.

Also disclosed are methods of increasing mammary gland development in Cobra-deficient subjects comprising administering a treatment that alters transcription of puberty-related genes, estrogen-responsive genes or progesterone-responsive genes. Altering transcription can be the increase or decrease in transcription.

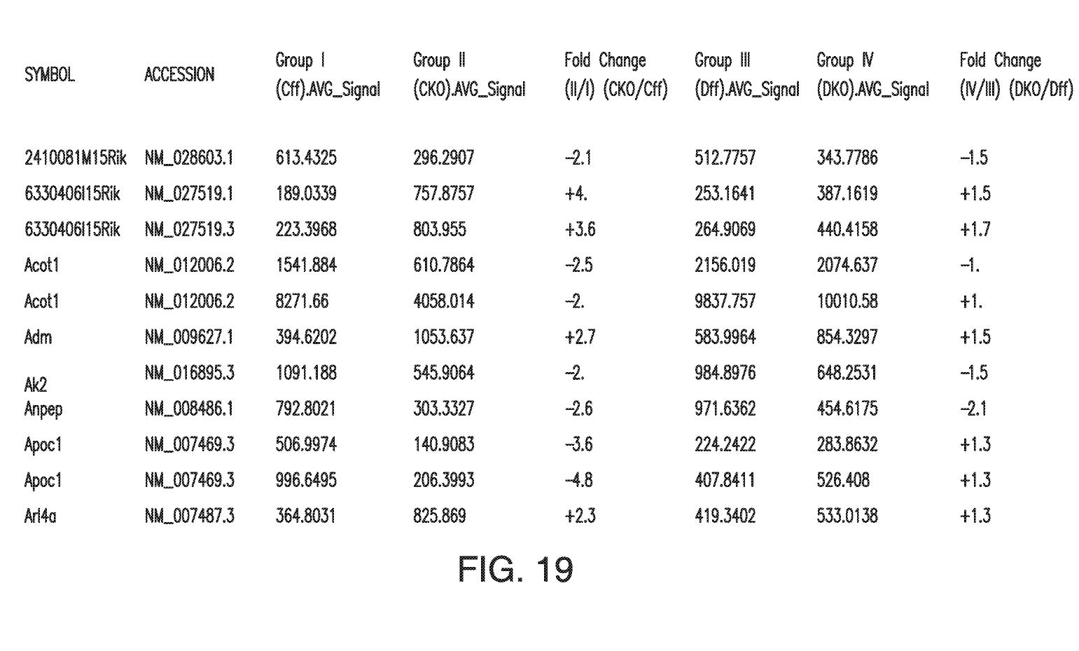

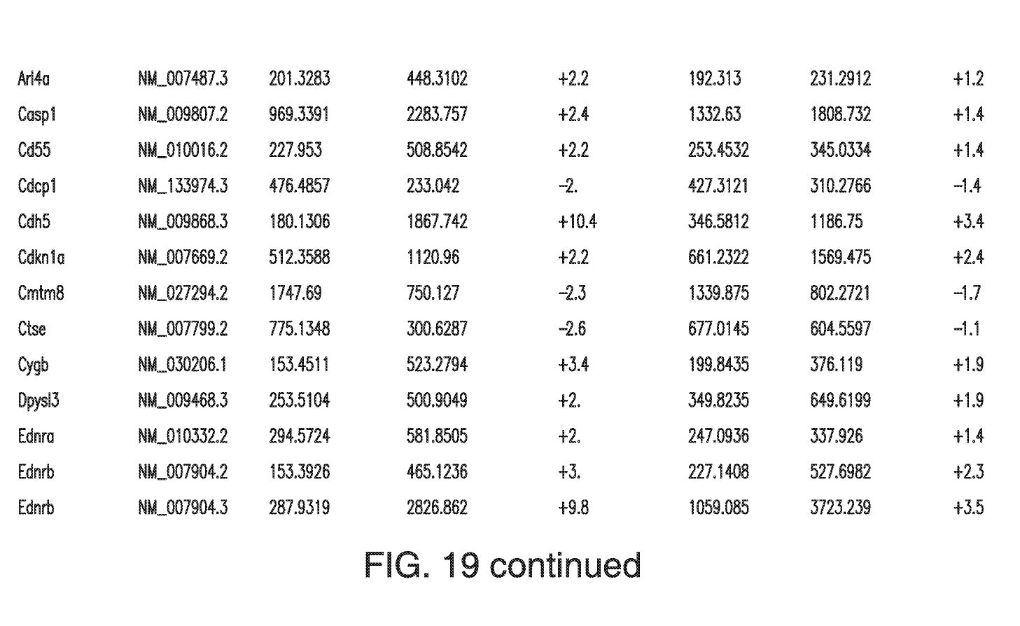

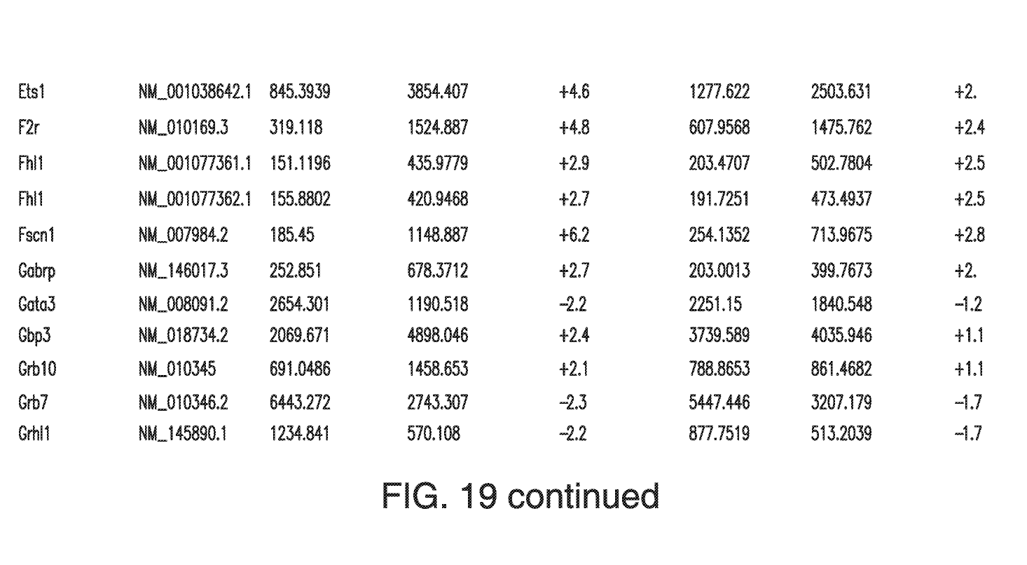

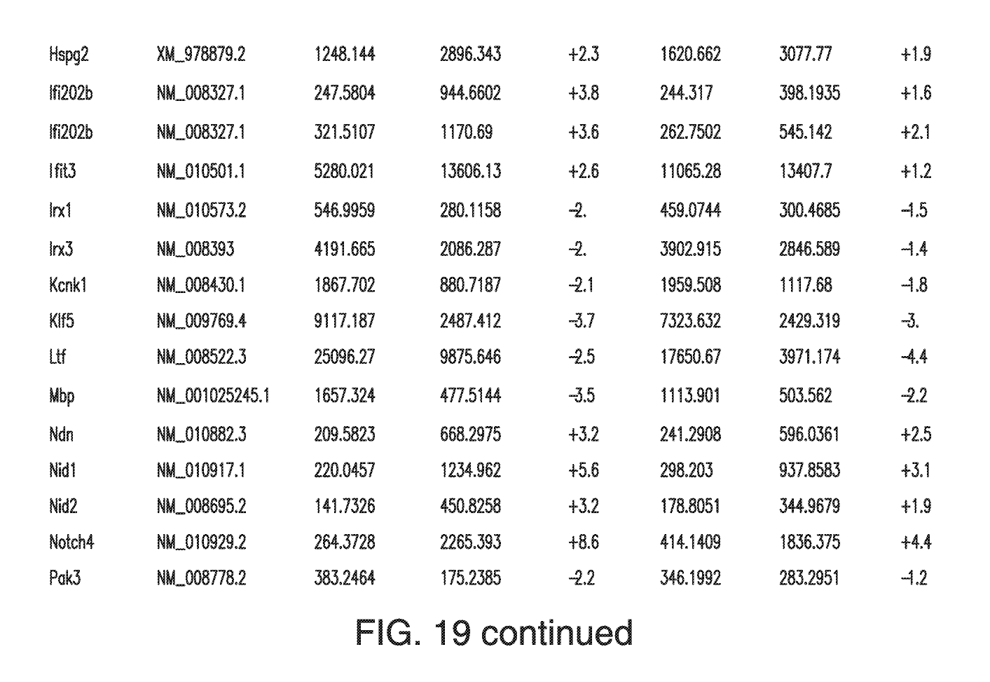

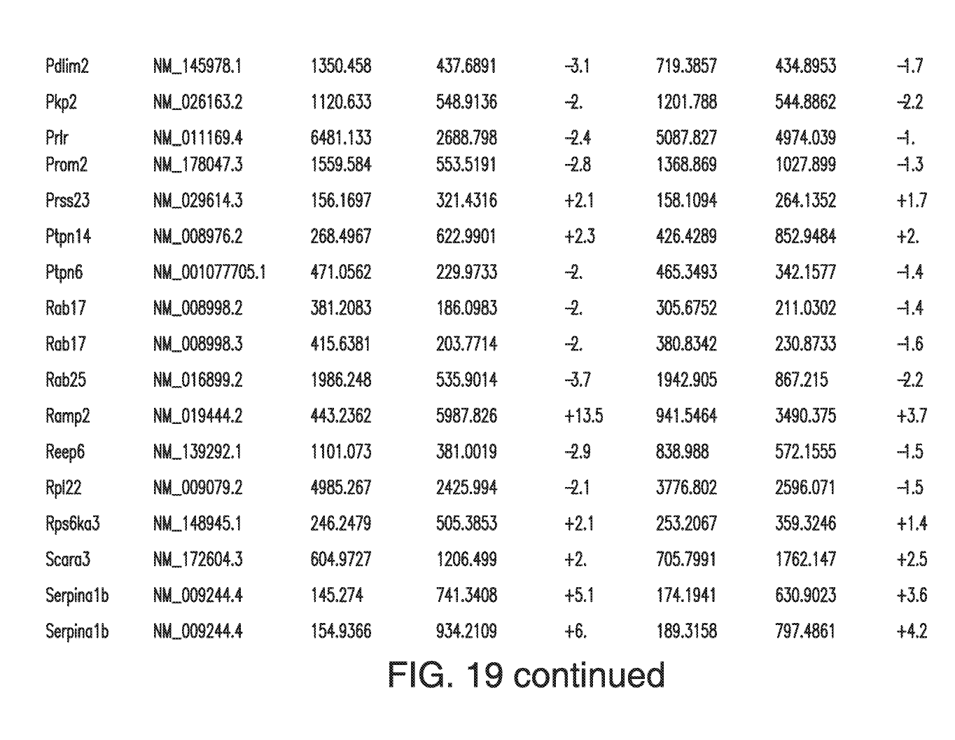

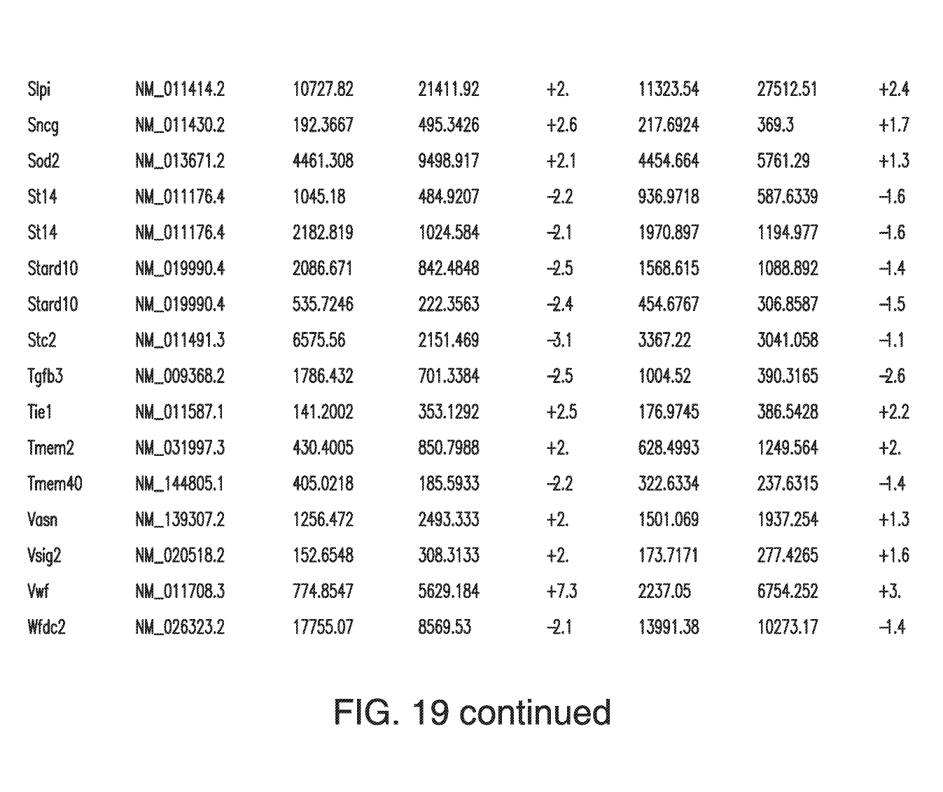

Disclosed are methods of increasing mammary gland development in Cobra-deficient subjects comprising administering a treatment that alters transcription of one or more puberty-related genes, estrogen-responsive genes or progesterone-responsive genes, wherein the puberty-related genes are Gata3, Prlr, Ramp2, Vwf, Prom2, or Acot1. Other puberty-related genes include, but are not limited to, those genes listed in FIG. 19.

Disclosed are methods of increasing mammary gland development in Cobra-deficient subjects comprising administering a treatment that alters transcription of one or more puberty-related genes, estrogen-responsive genes or progesterone-responsive genes, wherein the estrogen-responsive genes are 2410081M15RIK, 6430706D22RIK, ACOT1, ACTB, ARL4A, BCL6B, CTSH, CXCL9, EMCN, GBP6, GGTA1, HOXA7, PABPC1, PDLIM1, PDLIM2, PROM2, PTPN14, SLCO2B1, STARD10, TMEM2, WIPI1, or a combination thereof.

Disclosed are methods of increasing mammary gland development in Cobra-deficient subjects comprising administering a treatment that alters transcription of one or more puberty-related genes, estrogen-responsive genes or progesterone-responsive genes, wherein the progesterone-responsive genes are 5730593F17RIK, CCDC80, CDH13, CLDN5, CRYZ, EDN1, IRX1, NOXO1, PKP2, PRKCDBP, PSEN2, SLC7A3, SPNB3, or a combination thereof.

Disclosed are methods of increasing mammary gland development in Cobra-deficient subjects comprising administering a treatment that alters transcription of puberty-related genes, estrogen-responsive genes or progesterone-responsive genes, wherein a treatment that alters transcription comprises a treatment that increases transcription.

Disclosed are methods of increasing mammary gland development in Cobra-deficient subjects comprising administering a treatment that alters transcription of puberty-related genes, estrogen-responsive genes or progesterone-responsive genes, wherein a treatment that alters transcription comprises a treatment that decreases transcription.

### EXAMPLES

### G. BRCA1 Balances the Action of a Transcription Elongation Factor in Mammary Gland Development and Tumorigenesis

1. Introduction

Germ-line mutations in BRCA1 predispose women to breast and ovarian cancers. The preponderant association of BRCA1 tumors with female reproductive organs has been well established. Furthermore, the origin of BRCA1 breast tumors has been traced to progenitors of the luminal epithelial compartment, which constitutes the inner layer of breast epithelium. As BRCA1 protein is expressed in a variety of tissue and cell types outside breast and ovaries, context-dependent BRCA1 activity must underlie its sex and tissue-specific tumor suppressor function. However, the exact mechanism by which BRCA1 suppresses tumors in breast and ovaries remains poorly understood, even two decades after the cloning of the BRCA1 gene. The enduring conundrum of tissue specificity for tumor suppressor function is not limited to BRCA1. For example, until recently, it was unclear why germ-line mutations of the RB1 gene specifically predispose children to retinoblastoma. It was recently shown that the idiosyncratic signaling circuitry in cone progenitor cells renders these cells particularly sensitive to tumorigenesis initiated by RB1 loss. In another salient example, the product of tumor suppressor gene ATM plays a critical role in DNA damage response (DDR) to double-strand breaks (DSB). However, recent mouse genetic studies identified a DDR-independent function of ATM in modulating mitochondrial homeostasis, which could explain some of the clinical phenotypes associated with ataxia telangiectasia.

Mechanistically, BRCA1 is best known for its role in promoting the homologous recombination (HR)-based pathway of DSB repair. BRCA1 forms multi-protein complexes in response to DSBs and acts as a scaffolding protein to recruit various DNA repair proteins to the break sites, thus facilitating DNA repair per se and/or activating cell cycle checkpoints. Cancer-predisposing mutations of BRCA1 abolish its DSB repair activity, thus underscoring the clinical relevance of BRCA1 function in DSB repair. However, as such BRCA1 activity can be readily demonstrated in vitro using established cell lines that are not limited to breast and ovarian origins, it is unclear as to why the loss of a universal function of BRCA1 in DSB repair leads to highly sex/tissue-specific tumor development in vivo.

In addition to its well-documented role in DSB repair, BRCA1 has also been implicated in other cellular processes including transcriptional regulation and heterochromatin-mediated gene silencing. BRCA1 binds to RNA polymerase II (RNAPII) and various site-specific transcription factors including estrogen receptor α (ERα) and GATA3, which are involved in mammary gland development and breast cancer. Consistent with a role for BRCA1 in transcription-related events, genome-wide analysis indicates that chromatin binding of BRCA1 is enriched at transcription start sites (TSS) across the human genome. Notably, recent cell line-based studies also implicate BRCA1 in elimination of R loops, by-products of transcription. R loops consist of a DNA-RNA hybrid between nascent RNA and the template DNA strand, and an unpaired single-stranded DNA from the non-template strand. R loops have become an increasingly appreciated source of genetic instability and important regulators of transcription, DNA methylation, and chromatin architecture. Given the divergent roles of R loops in genome integrity and gene expression, prevention of R-loop accumulation by BRCA1 could suppress cancer development via multiple mechanisms. Notwithstanding these in vitro findings, compelling in vivo evidence for the importance of these transcription-related activities of BRCA1 to BRCA1-mediated tumor suppression is lacking.

A BRCA1-binding protein, cofactor of BRCA1 or COBRA1, which is identical to the B subunit of the four-subunit NELF complex (NELF-B) was previously identified. NELF is a metazoan-specific transcription elongation factor that pauses RNAPII at a TSS-proximal region. Although NELF was first identified as an transcription elongation repressor in vitro, subsequent in vivo studies indicate that NELF-mediated RNAPII pausing can lead to decreased or increased transcription. In ERα+ breast cancer cells, COBRA1/NELF-B interacts with ERα and regulates RNAPII movement at ERα target genes. While NELF-mediated RNAPII pausing has been proposed to ensure synchronous transcriptional activation of developmentally regulated genes, the exact physiological roles of mammalian NELF have just begun to be deciphered. Mouse COBRA1/NELF-B is critical for early embryogenesis and energy homeostasis in adult myocardium.

Using mammary epithelium-specific knockout (KO) mouse models for Brca1 and Cobra1, the functional relationship between these two genes in mammary gland morphogenesis and tumorigenesis was investigated. A tissue-selective and DSB repair-independent functional interaction between Brca1 and Cobra1 was identified. This previously unappreciated balancing act between BRCA1 and a key transcription elongation factor promotes normal tissue development while suppressing tumorigenesis in the same mammary epithelial compartment.

2. Results

i. Genetic Complementation between Brca1 and Cobra1 in Mammary Gland Development and Function

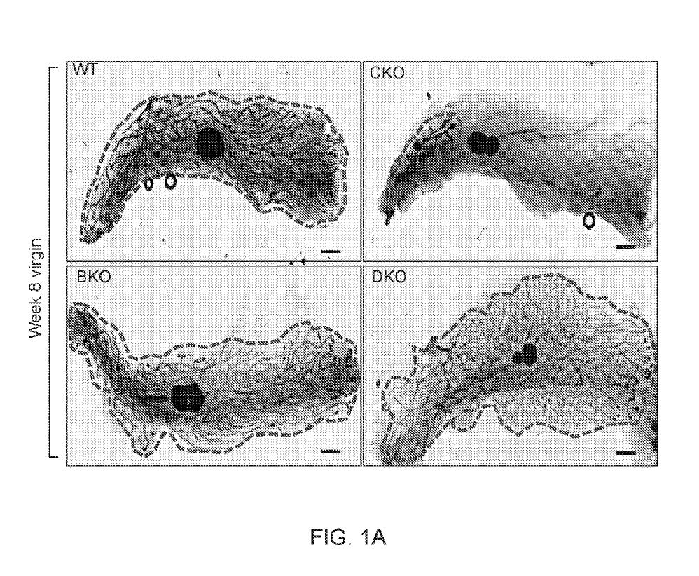

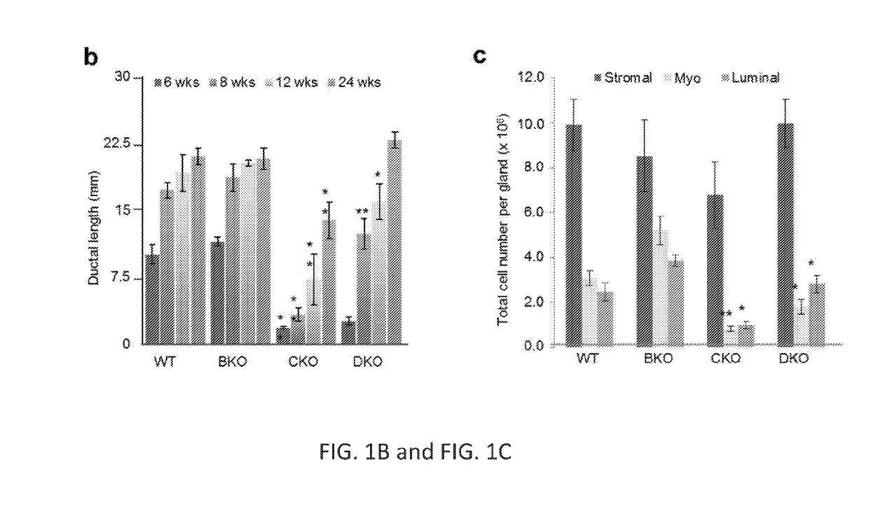

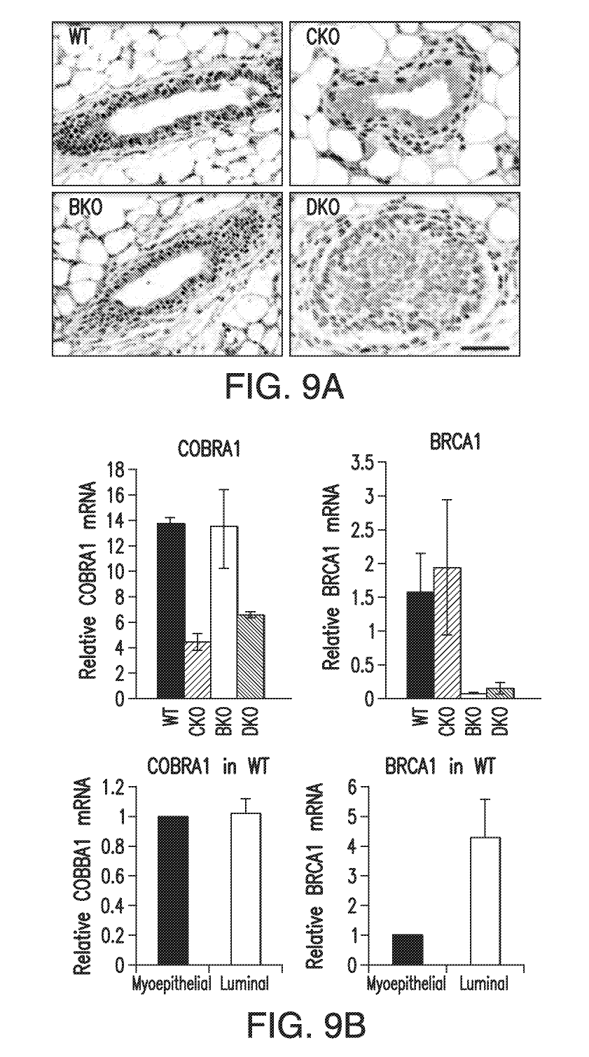

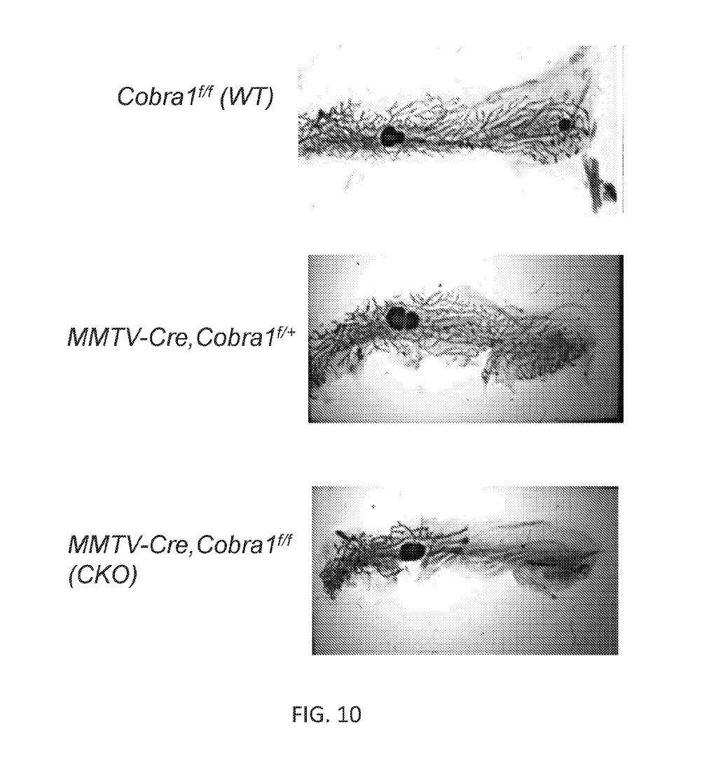

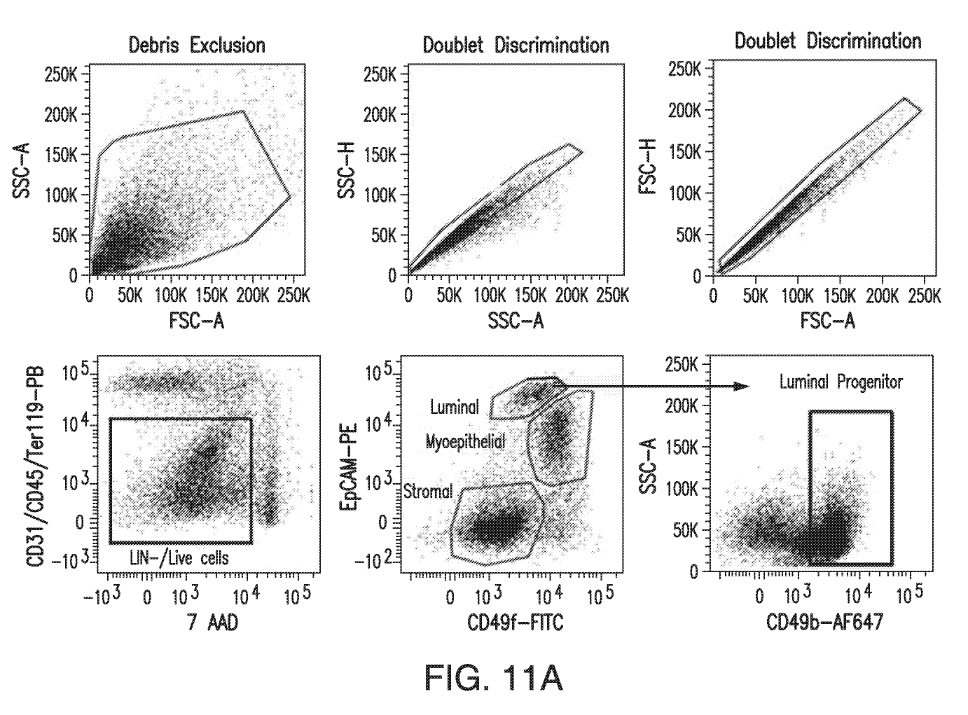

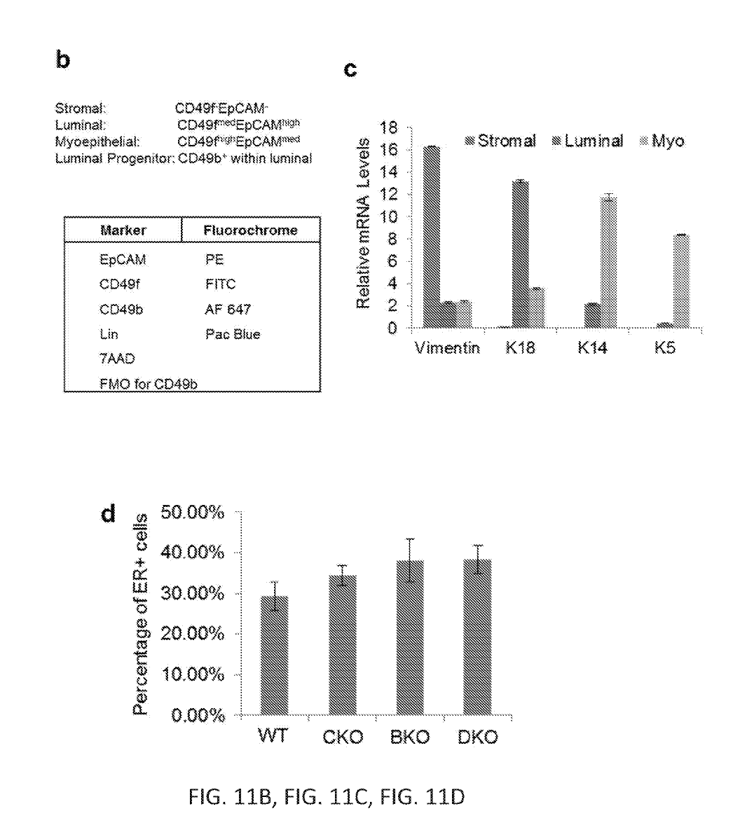

To investigate the role of COBRA1 in mammary gland development, mammary epithelium-specific KO mice were generated by breeding the MMTV-Cre strain with Cobra1f/f animals that resulted in deletion of the first 4 Cobra1 exons. Mammary epithelium of the resulting female MMTV-Cre,Cobra1f/f (CKO) animals was effectively depleted of COBRA1 mRNA and protein (FIG. 9). Compared with age-matched wild-type (WT, Cobra1f/f) and hemizygous mice (MMTV-Cre,Cobra1f/+), CKO with homozygous deletion of Cobra1 displayed severely retarded mammary ductal growth (FIG. 1a,b and FIG. 10). The developmental defect of CKO was most profound during and shortly after puberty (6 and 8 weeks), and remained significant in older virgin mice (12 and 24 weeks, FIG. 1b). In further support, flow cytometry using established cell surface markers for mammary epithelial cells65 showed that luminal (CD49fmedEpCAMhigh) and myoepithelial (CD49fhighEpCAMmed) cell populations of CKO mammary glands were equally reduced compared to WT controls (FIG. 1c and FIG. 11a-c). Furthermore, the CKO luminal compartment did not exhibit any significant change in the relative abundance of ER+ over ER− cells (FIG. 11d), consistent with an overall developmental arrest of multiple mammary epithelial lineages.

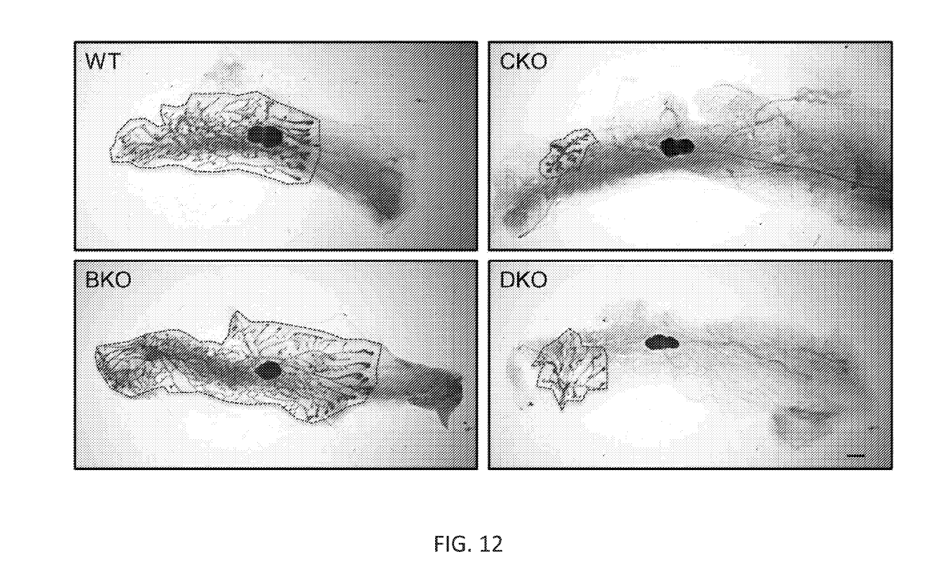

Given the physical interaction between BRCA1 and COBRA1, the phenotypes of CKO were compared with MMTV-Cre-mediated Brca1 KO that was conditionally deleted of Brca1 exon 11 (MMTV-Cre,Brca1f/f; BKO), and Brca1/Cobra1 double KO mice (MMTV-Cre,Brca1f/f,Cobra1f/f; DKO). BKO animals exhibited normal ductal growth at puberty (FIG. 1a-b). Ductal development of DKO mice was stunted at 6 weeks (FIG. 1b and FIG. 12), but remarkably, it approached that of WT and BKO at later stages (FIG. 1a,b). Furthermore, the abundance of luminal and myoepithelial cells in DKO mammary glands was largely restored to WT levels (FIG. 1c). COBRA1 and BRCA1 expression in DKO mice were depleted to a similar extent versus the corresponding single-gene KO animals (FIG. 9). Therefore the marked phenotypic difference between CKO and DKO reflects a bona fide genetic complementation between Brca1 and Cobra1. CKO mammary glands manifest a Brca1-dependent developmental blockade.

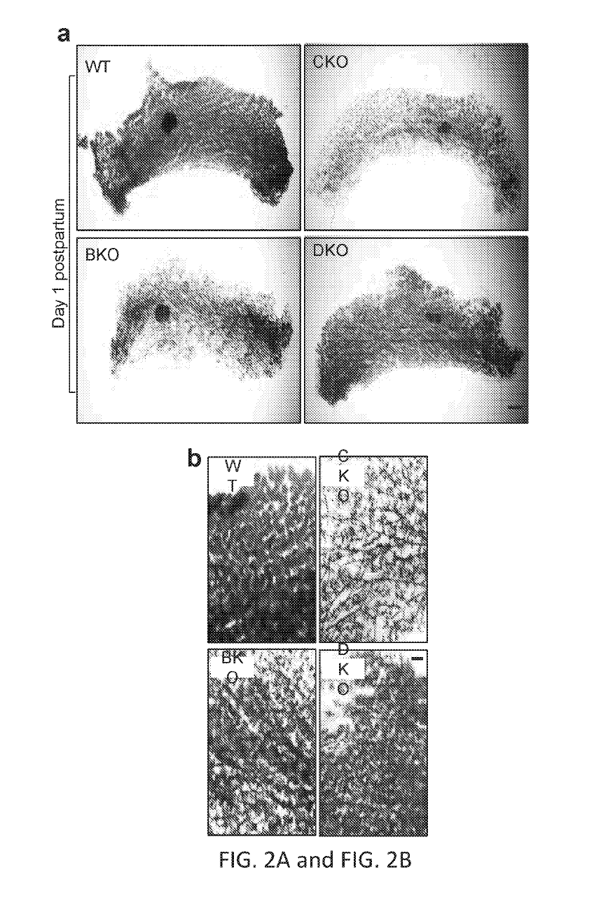

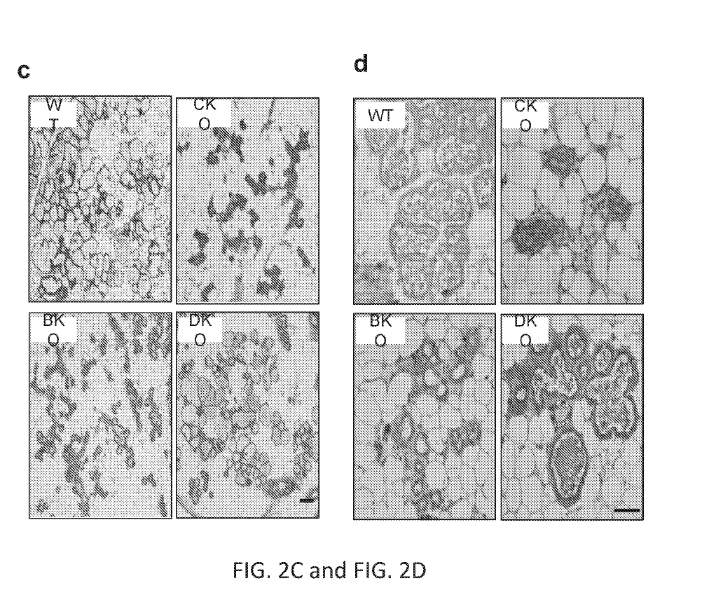

Despite the partial ductal growth in older virgin CKO (FIG. 1b), all pups of CKO dams died shortly after birth from obvious lack of nursing. In support, mammary glands of CKO at postpartum were largely devoid of alveolar structure (FIG. 2a-c) and milk proteins (FIG. 2d). Similar, albeit less severe, alveologenic and lactogenic defects were observed in BKO mammary glands (FIG. 2). In stark contrast, DKO dams with simultaneous deletion of Brca1 and Cobra1 underwent efficient alveologenesis and lactogenesis, as evidenced by the normal alveolar structure (FIG. 2a-c), abundant milk proteins (FIG. 2d), and restored nursing ability. Collectively, these genetic data unequivocally demonstrate a functional interaction between Brca1 and Cobra1 in mammary gland development and function.

ii. The Brca1 and Cobra1 Genetic Interaction is Specific for Mammary Glands

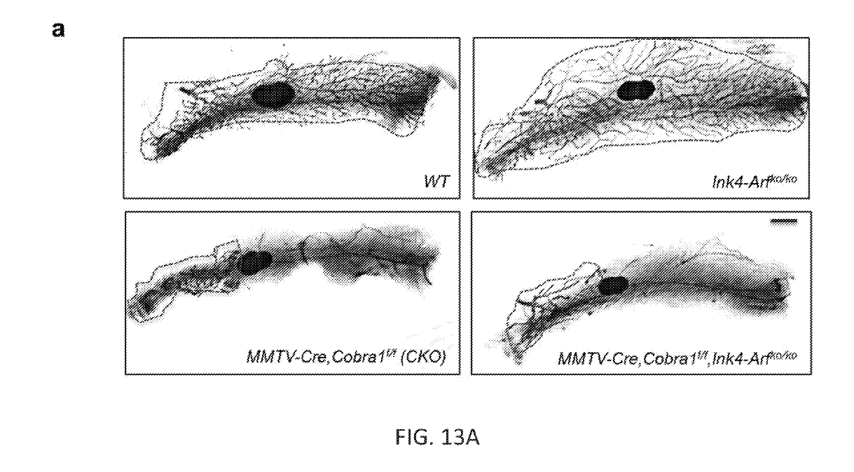

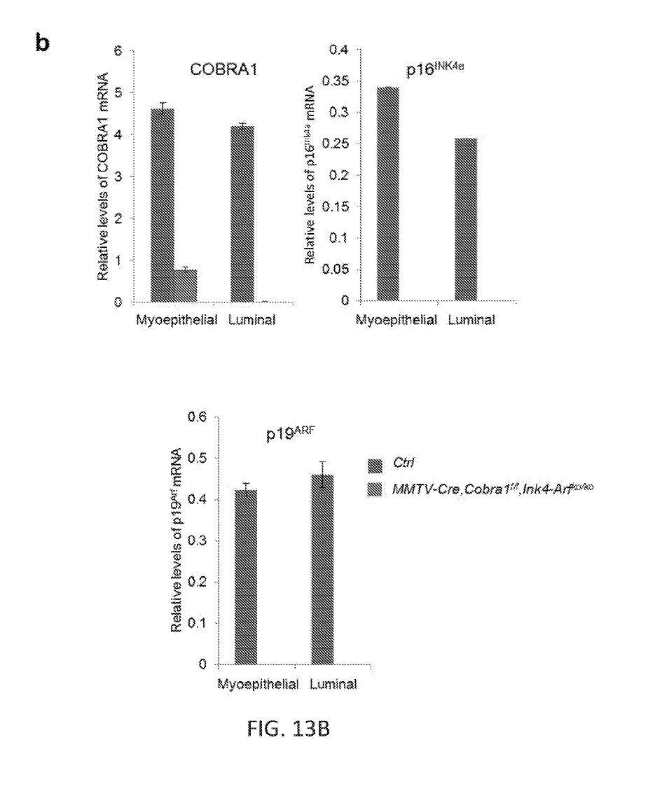

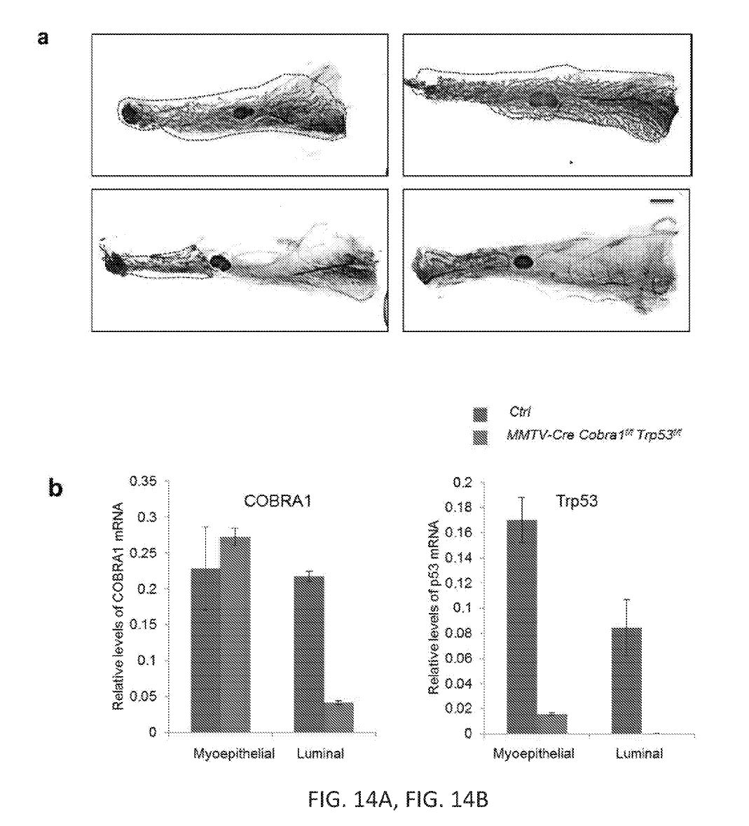

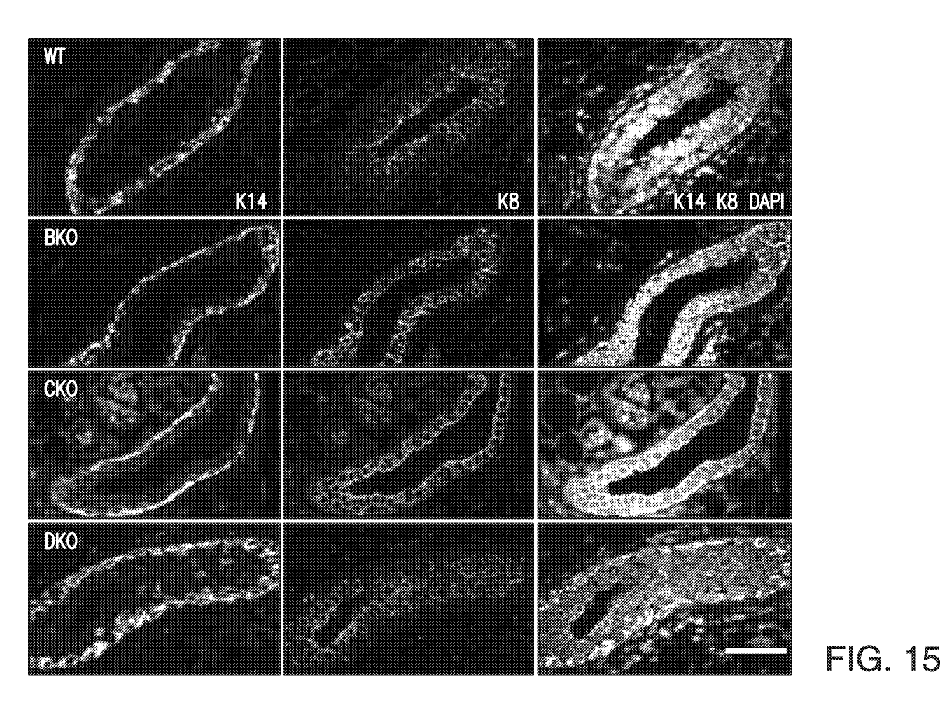

To determine how specific the genetic complementation is between Brca1 and Cobra1, whether genetic ablation of other growth-arresting tumor suppressor genes could rescue the developmental defects associated with CKO was investigated. Tumor suppressor genes Ink4-Arf play a critical role in oncogene-induced senescence, and co-deletion of the Ink4a/Arf locus restored developmental defect associated with the loss of Bmi1, a transcriptional regulator of stem cell renewal. In addition, deletion of tumor suppressor gene Trp53 partially rescued early embryonic lethality associated with Brca1 deficiency. CKO was combined with whole-body deletion of Ink4-Arf or mammary gland-specific deletion of Trp53. In contrast to Brca1 deletion, neither Ink4-Arf nor Trp53 deficiency rescued the ductal growth defect of CKO (FIGS. 13 and 14), indicating a specific genetic interaction between Brca1 and Cobra1.

Whether the genetic complementation between Brca1 and Cobra1 was tissue-dependent was investigated. Homozygous deletion of Brca1 or Cobra1 causes early embryonic lethality. Mice that carried hemizygous germ-line deletions of Brca1 and Cobra1 (Brca1+/−Cobra1+/−) were bred and progenies were examined for rescue of embryonic lethality. Upon genotyping a large number of embryos and viable pups, we did not find any with homozygous deletion of both genes (Brca1−/−,Cobra1−/−, Table 1). Taken together, these findings indicate a tissue and gene-selective genetic interaction between Cobra1 and Brca1 in mammary epithelium.

TABLE 1
Lethality of Brca1- and Cobra1-deleted embryos cannot be mutually
rescued by double KO

| Brca1, Cobra1 | FEMALE | MALE | F + M |
|---|---|---|---|
| +/+, +/+ | 11 | 20 | 31 |
| +/+, +/− | 25 | 18 | 43 |
| +/+, −/− | 0 | 0 | 0 |
| +/−, +/+ | 35 | 23 | 58 |
| +/−, +/− | 69 | 55 | 124 |
| +/−, −/− | 0 | 0 | 0 |
| −/−, +/− | 0 | 0 | 0 |
| −/−, −/− | 0 | 0 | 0 |
| TOTAL | 140 | 116 | 256 |

i. BRCA1 Inhibits Pubertal Transcription in COBRA1-Deficient Mammary Epithelium

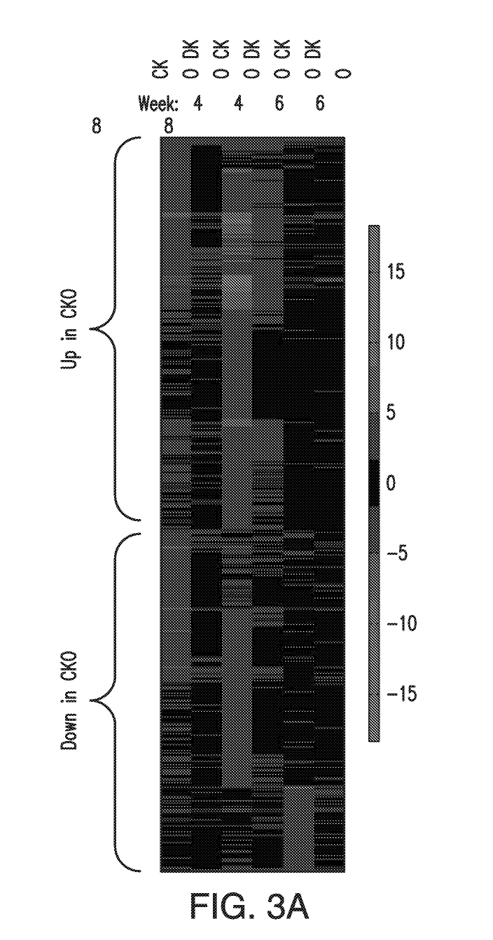

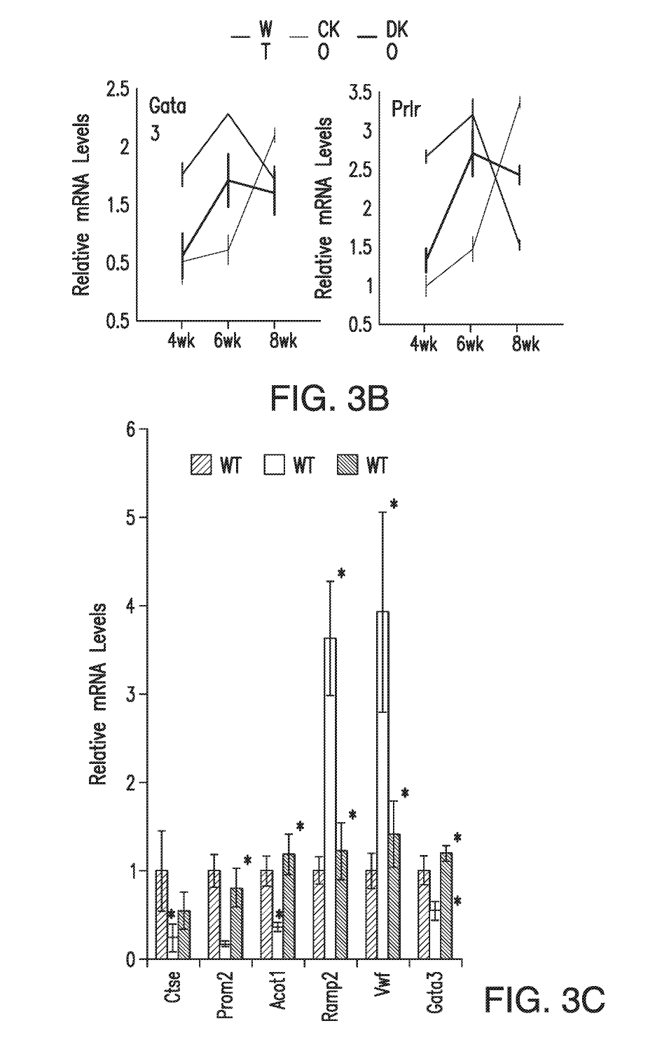

To gain molecular insight into the Brca1/Cobra1 genetic complementation during ductal development at puberty, gene expression profiling of sorted mammary epithelial cells from WT, BKO, CKO, and DKO at 4, 6, and 8 weeks was performed. Consistent with their normal ductal growth (FIG. 1a,b) and previously reported gene expression profiling of the same animal model, BKO mice exhibited very few transcriptionally affected genes compared to their WT controls (Table 2) and therefore were not included in the subsequent bioinformatics analysis. In contrast, the gene expression profiles of CKO were significantly different from their WT littermates, with the most significant transcriptional aberration observed at the early (4 week) and mid-pubertal (6 week) stages (FIG. 3 and Table 2). Furthermore, these CKO-affected genes were enriched with previously identified pubertal genes (P=7.65×10-13 for 6-wk), and estrogen (P=7.73×10-6) and progesterone-responsive genes (P=5.00×10-5) in mammary epithelium (Table 3). Strikingly, approximately 80% of the CKO-affected genes at 4 and 6 weeks were either partially or completely rescued in DKO mammary glands (FIG. 3 and Table 3). Likewise, the DKO-rescued genes were enriched with puberty-related (P=2.34×10-9 for 6-wk) and estrogen (P=2.09×10-5) and progesterone-responsive genes (P=7.64×10-4, Table 3). For example, expression of Gata3 and Prlr, two known pubertal genes, was disrupted by Cobra1 ablation but partially restored in DKO (FIG. 3b). The microarray result was confirmed for several pubertal genes by gene-specific RT-PCR (FIG. 3c). Of note, while the transcriptional rescue in DKO occurred as early as 4 weeks (FIG. 3a), restoration of ductal growth in DKO was not apparent until 8 weeks (FIG. 1a,b and FIG. 12), likely due to incomplete transcriptional rescue of CKO-affected genes. The fact that transcriptional rescue precedes developmental rescue indicates that the former is likely a cause, rather than consequence, of the restored ductal morphogenesis. Collectively, the data define an inhibitory activity of BRCA1 in pubertal transcription and ductal development that manifests in the absence of COBRA1.

TABLE 2
8 wks DKO affected gene list (DKO/WT ≥2 or DKO/WT ≤−2, p < 0.05)

| | | | | Fold |
|---|---|---|---|---|
| | | | | Change |
| | | Group I | Group II | (II/I) |
| SYMBOL | ACCESSION | (Dff).AVG_Signal | (DKO).AVG_Signal | (DKO/Dff) |
| Muc 1 | NM_013605.1 | 824.4185 | 206.1236 | −4.00 |
| Bglap-rs1 | NM_031368.3 | 492.883 | 126.5445 | −3.89 |
| Dmkn | NM_172899.3 | 1873.177 | 579.8171 | −3.23 |
| Muc 1 | NM_013605.1 | 912.9594 | 284.6109 | −3.21 |
| Clic6 | NM_172469.3 | 709.8447 | 222.9155 | −3.18 |
| Dmkn | NM_172899.2 | 1955.163 | 626.6171 | −3.12 |
| Muc 1 | NM_013605.1 | 523.3958 | 172.2742 | −3.04 |
| Muc 1 | NM_013605.1 | 403.5068 | 135.7065 | −2.97 |
| C3 | NM_009778.1 | 1956.615 | 661.4249 | −2.96 |
| Scara5 | NM_028903.1 | 557.677 | 191.1359 | −2.92 |
| Trf | NM_133977.2 | 7689.115 | 2657.633 | −2.89 |
| AI428936 | NM_153577.2 | 613.7659 | 228.6717 | −2.68 |
| Cd14 | NM_009841.3 | 5180.815 | 1935.141 | −2.68 |
| Slc13a2 | NM_022411.3 | 259.2089 | 100.97 | −2.57 |
| Gdpd3 | NM_024228.2 | 1361.444 | 536.8906 | −2.54 |
| Ltf | NM_008522.3 | 21105.27 | 8340.986 | −2.53 |
| Ceacam1 | NM_001039187.1 | 803.8411 | 318.2288 | −2.53 |
| Elf5 | NM_010125.2 | 718.727 | 285.1689 | −2.52 |
| Ogfrl1 | NM_001081079.1 | 732.4705 | 297.2078 | −2.46 |
| Cyp24a1 | NM_009996.2 | 278.1577 | 115.2921 | −2.41 |
| Cyp2d22 | NM_019823.3 | 813.0899 | 338.1125 | −2.40 |
| AU040829 | NM_175003.3 | 432.2578 | 188.2646 | −2.30 |
| Btn1a1 | NM_013483.2 | 351.197 | 161.8884 | −2.17 |
| A730008L03Rik | NM_021393.1 | 311.523 | 144.6753 | −2.15 |
| Ltf | NM_008522.2 | 349.789 | 166.111 | −2.11 |
| Ckmt1 | NM_009897.2 | 523.3627 | 249.3914 | −2.10 |
| Pabpc1 | NM_008774.2 | 2434.311 | 1162.352 | −2.09 |
| Bglap1 | NM_001037939.1 | 220.6627 | 105.7095 | −2.09 |
| Gjb2 | NM_008125.2 | 627.1307 | 304.1209 | −2.06 |
| Elf5 | NM_010125.2 | 288.463 | 140.1559 | −2.06 |
| Xbp1 | NM_013842.2 | 7508.566 | 3669.257 | −2.05 |
| A730008L03Rik | NM_021393.1 | 298.9673 | 146.1672 | −2.05 |
| Atf5 | NM_030693.1 | 940.6545 | 462.3745 | −2.03 |
| Tmc4 | NM_181820.2 | 987.8871 | 488.3747 | −2.02 |
| Igfbp5 | NM_010518.2 | 8533.547 | 4231.91 | −2.02 |
| Slc5a8 | NM_145423.2 | 2351.473 | 1170.86 | −2.01 |
| Scd1 | NM_009127.3 | 1553.969 | 776.8299 | −2.00 |
| Ceacam1 | NM_001039185.1 | 355.335 | 177.758 | −2.00 |
| Krt79 | NM_146063.1 | 103.8764 | 207.4121 | +2.00 |
| Lgmn | NM_011175.2 | 534.8884 | 1069.188 | +2.00 |
| Hist1h3d | NM_178204.1 | 220.1151 | 441.7187 | +2.01 |
| Psmb9 | NM_013585.2 | 118.0195 | 239.2073 | +2.03 |
| Hist1h4f | NM_175655.1 | 180.4794 | 368.4062 | +2.04 |
| Hist1h2bn | NM_178201.1 | 385.2098 | 796.5076 | +2.07 |
| Hdc | NM_008230.4 | 133.3217 | 279.6878 | +2.10 |
| Cenpa | NM_007681.2 | 631.3411 | 1326.15 | +2.10 |
| Hist1h3e | NM_178205.1 | 223.9645 | 473.4436 | +2.11 |
| Fb1n2 | NM_001081437.1 | 199.1063 | 421.028 | +2.11 |
| Ccl5 | NM_013653.2 | 316.7475 | 670.5001 | +2.12 |
| Hist1h2bj | NM_178198.1 | 1375.097 | 2918.971 | +2.12 |
| Hist1h1c | NM_015786.1 | 3375.41 | 7220.594 | +2.14 |
| Rbp7 | NM_022020.2 | 102.1815 | 221.7561 | +2.17 |
| Itih2 | NM_010582.2 | 139.8296 | 307.2296 | +2.20 |
| Hist1h2bf | NM_178195.1 | 1180.516 | 2613.672 | +2.21 |
| Cxcl9 | NM_008599.3 | 114.0531 | 252.5468 | +2.21 |
| Hist1h4m | NM_175657.1 | 132.5918 | 296.3 | +2.23 |
| Cited1 | NM_007709.3 | 187.3248 | 420.7397 | +2.25 |
| Ly6c1 | NM_010741.2 | 283.9633 | 649.633 | +2.29 |
| Lgals7 | NM_008496.4 | 273.0249 | 626.1846 | +2.29 |
| Hist1h2bc | NM_023422.3 | 953.8706 | 2222.036 | +2.33 |
| Cdkn1a | NM_007669.2 | 194.9874 | 458.4763 | +2.35 |
| Hist1h2bh | NM_178197.1 | 697.3153 | 1647.799 | +2.36 |
| Vim | NM_011701.3 | 219.9753 | 528.9696 | +2.40 |
| EG630499 | NM_001081015.1 | 127.463 | 309.3895 | +2.43 |
| Hist1h2bk | NM_175665.1 | 466.8949 | 1146.251 | +2.46 |
| Cdkn1a | NM_007669.3 | 605.1024 | 1485.854 | +2.46 |
| Hist1h4k | NM_178211.1 | 139.4921 | 348.1631 | +2.50 |
| Cav1 | NM_007616.3 | 465.0926 | 1161.802 | +2.50 |
| Cdkn1a | NM_007669.2 | 264.1147 | 670.9604 | +2.54 |
| Actg2 | NM_009610.1 | 804.6465 | 2051.867 | +2.55 |
| Aqp1 | NM_007472.1 | 142.4449 | 372.2909 | +2.61 |
| Selp | NM_011347.1 | 322.0848 | 842.3918 | +2.62 |
| Ccl21c | NM_023052.1 | 158.7866 | 425.8169 | +2.68 |
| Cst3 | NM_009976.3 | 934.4656 | 2530.607 | +2.71 |
| 2210407C18Rik | NM_144544.1 | 228.2843 | 618.973 | +2.71 |
| Dnaic1 | NM_175138.3 | 149.6938 | 415.3302 | +2.77 |
| Hist1h4i | NM_175656.2 | 173.086 | 520.6535 | +3.01 |
| Plvap | NM_032398.1 | 180.8246 | 551.1104 | +3.05 |
| Cxcl10 | NM_021274.1 | 251.0965 | 768.627 | +3.06 |
| Serpinf1 | NM_011340.3 | 262.5768 | 822.2831 | +3.13 |
| H2-T23 | NM_010398.1 | 693.5092 | 2233.837 | +3.22 |
| Mmrn2 | NM_153127.3 | 150.8225 | 492.4206 | +3.26 |
| Aqp1 | NM_007472.2 | 226.3429 | 758.06 | +3.35 |
| Ckb | NM_021273.3 | 720.8067 | 2427.159 | +3.37 |
| Hist2h2aa1 | NM_013549 | 158.6823 | 585.7575 | +3.69 |
| Hist1h4j | NM_178210.1 | 171.9211 | 651.2156 | +3.79 |
| Ccl21a | NM_011124.4 | 245.4477 | 945.769 | +3.85 |
| Upk3a | NM_023478.1 | 171.1022 | 685.8395 | +4.01 |
| Vwf | NM_011708.3 | 253.2617 | 1033.758 | +4.08 |
| Hist1h4h | NM_153173.2 | 151.452 | 662.3843 | +4.37 |

TABLE 3
Overlap between CKO-affected genes and published gene list. Number in
parentheses is p-value of the overlap calculated by Fisher's exact test.

| | Gene | Pubertal | Estrogen | Progesterone |
|---|---|---|---|---|
| | count | 781 | 195 | 118 |
| 4 wk affected | 323 | 43 (4.52E−11) | 9 (0.008) | 5 (0.060) |
| 6 wk affected | 600 | 67 (7.65E−13) | 19 (7.73E−06) | 12 (0.0005) |
| 6 wks rescued | 488 | 55 (2.34E−9) | 18 (2.09E−5) | 11 (7.62E−4) |
| 8 wk affected | 145 | 28 (3.40E−12) | 6 (0.005) | 2 (0.24) |

ii. COBRA1 Contributes to BRCA1-Associated Mammary Tumorigenesis

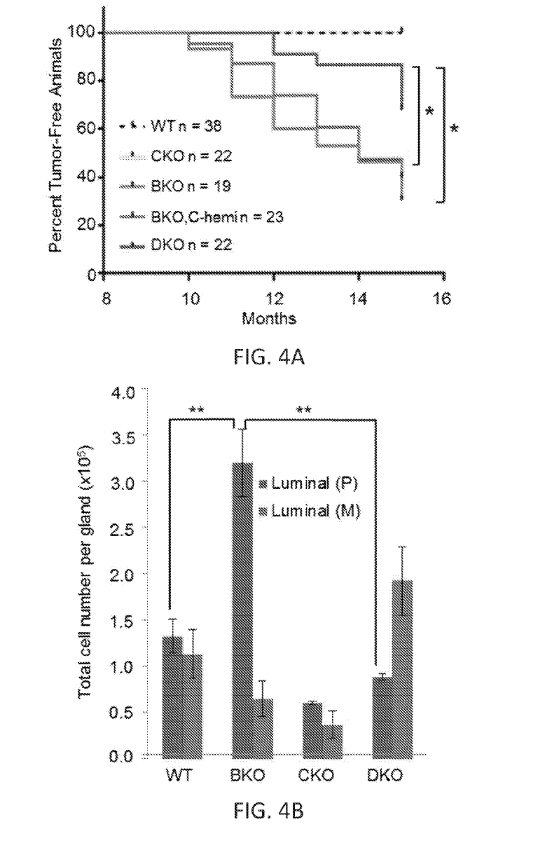

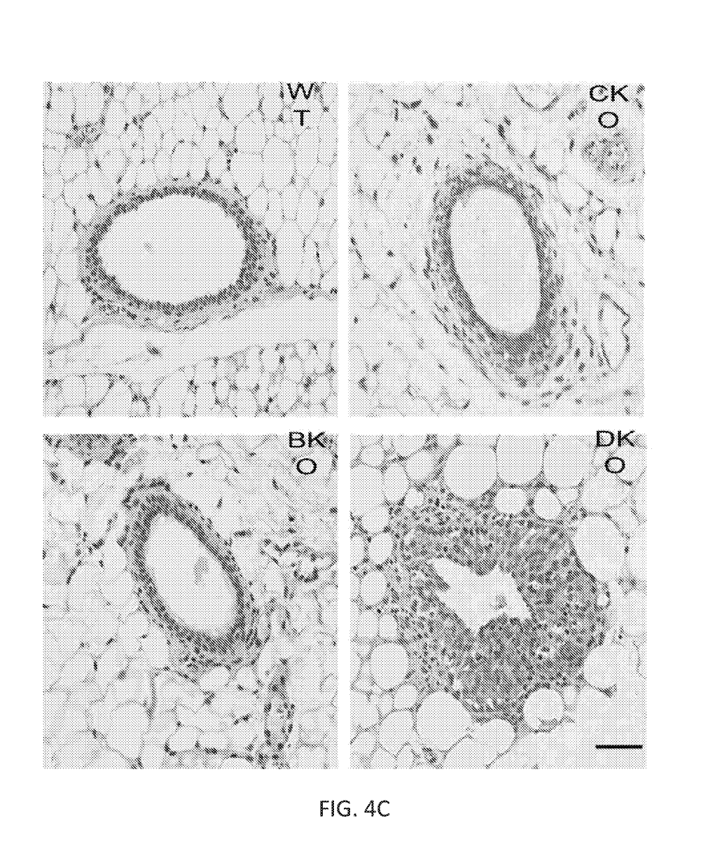

Given the roles of Brca1 in mediating the developmental arrest and transcriptional changes in Cobra1-deficient mammary glands, the reciprocal question of whether Cobra1 could influence mammary tumor development in Brca1-deficient mice was investigated. CKO mice did not display elevated mammary tumor occurrence versus WT control (FIG. 4a). Consistent with published findings, BKO mice had increased spontaneous mammary tumors (FIG. 4a). Hemizygous deletion of Cobra1 in the BKO background (BKO,C-hemi) did not affect Brca1-associated tumorigenesis (FIG. 4a). In stark contrast, DKO mice exhibited significantly lower incidence of tumorigenesis than BKO and BKO,C-hemi mice (FIG. 4a), indicating that Cobra1 deletion mitigated Brca1-associated tumorigenesis. Thus, COBRA1 can exacerbate mammary tumor development in the absence of functional BRCA1.

Consistent with the notion that luminal progenitor cells are the cell of origin for BRCA1-associated breast tumors, it was found that BKO had more luminal epithelial cells with CD49b expression, an established marker for the luminal progenitor population, compared to WT animals (FIG. 4b and FIG. 11). In contrast, CKO mammary glands contained markedly reduced pools of both mature (CD49b−) and progenitor (CD49b+) cells in the luminal epithelial compartment (FIG. 4b), again indicating inhibition of mammary epithelial cells of all lineages and differentiation stages upon Cobra1 ablation. Intriguingly, the flow cytometry profiles of DKO were distinct from those of BKO and CKO. In particular, the luminal progenitor cell population in DKO remained substantially lower than that in BKO (FIG. 4b), yet the mature luminal cell population in DKO was more abundant compared to BKO and CKO (FIG. 4b). Notably, DKO mammary ducts tended to have thickened epithelial layers (FIG. 4c), which were contributed predominantly by cells with known luminal markers keratin 8 and 18 (K8 and K18) (FIG. 15). Thus, excessive differentiation into mature cells of the same lineage could result in reduced pools of luminal progenitor cells in DKO mammary glands, which offers an explanation at the cellular level for the lower incidence of tumor development in DKO mice.

iii. The Effect of COBRA1 on BRCA1-Associated Tumorigenesis is Independent of DSB Repair

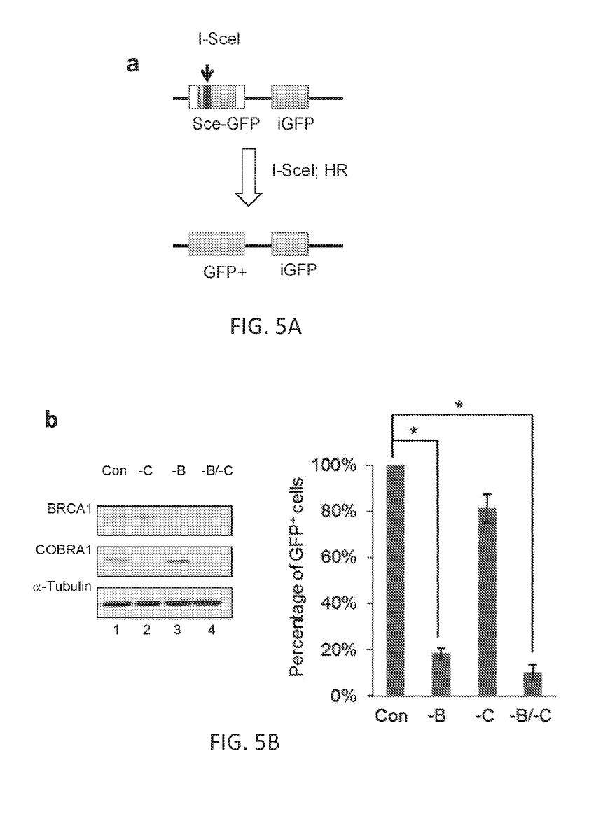

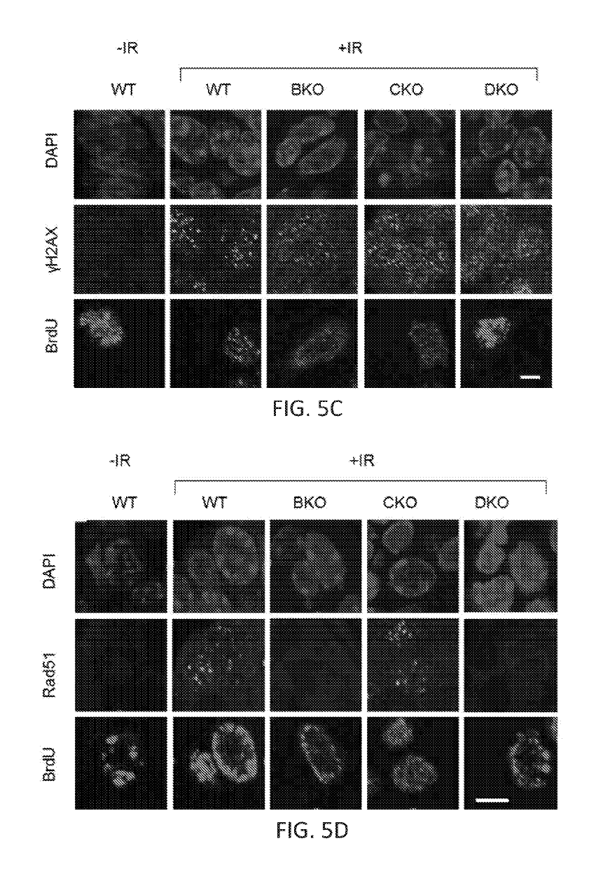

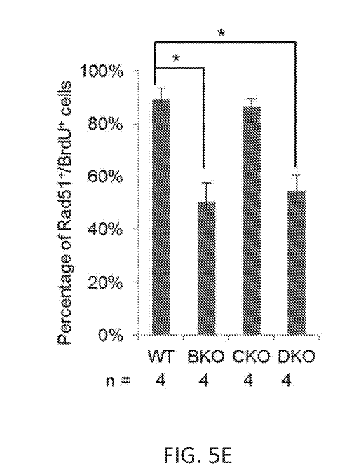

The genetic interaction between Brca1 and Cobra1 is reminiscent of the previously reported antagonism between Brca1 and 53BP1, whereby loss of 53BP1 eliminates BRCA1-associated mammary tumorigenesis and rescues HR-mediated DSB repair in BRCA1-deficient cells. Whether reduced tumorigenesis in DKO mice could be due to restored HR-mediated DSB repair was investigated. A green fluorescence protein (GFP)-based reporter assay was used in vitro, in which repair of site-specific DSB through the HR-dependent pathway gives rise to a functional GFP gene (FIG. 5a). As expected, BRCA1 knockdown significantly compromised HR efficiency, as indicated by reduced GFP+ cell numbers (FIG. 5b). Depletion of COBRA1 alone did not affect HR efficiency, nor did it rescue the HR defect in BRCA1-depleted cells (FIG. 5b), suggesting that COBRA1 did not affect BRCA1-mediated DSB repair in vitro.

Next, HR efficiency was examined in vivo following ionizing radiation (IR). HR repair predominantly occurs in proliferating cells during late S and G2 phases of the cell cycle, when sister chromatids are available as the homologous templates for HR-mediated repair. Proliferating cells were tracked in irradiated mice by pulse-labeling them with bromodeoxyuridine (BrdU). DSB damage was monitored 3 hours after irradiation by immunofluorescence staining for γH2AX. As expected, IR-induced γH2AX nuclear foci were present in both BrdU+ and BrdU− cells of WT and KO animals (FIG. 5c). To assess efficiency of HR-dependent DSB repair, BrdU+ mammary epithelial cells with IR-induced nuclear foci of Rad51, an HR marker, recruitment of which to DSB sites is facilitated by BRCA1 were enumerated. Consistent with the well-established role of BRCA1 in HR, irradiated BKO animals exhibited substantially lower Rad51+/BrdU+ ratios versus WT (FIG. 5d,e). CKO mammary glands had similar Rad51+/BrdU+ ratios versus WT control, indicating that COBRA1 per se is not involved in IR-induced DSB repair. Notably, HR repair in DKO mice remained as deficient as that in BKO (FIG. 5d,e). Taken together, both in vitro and in vivo data clearly indicate that the reduced mammary tumorigenesis in DKO versus BKO is not due to restored HR-mediated DSB repair.

iv. COBRA1 Promotes R-Loop Accumulation in BRCA1-Deficient Mammary Epithelium

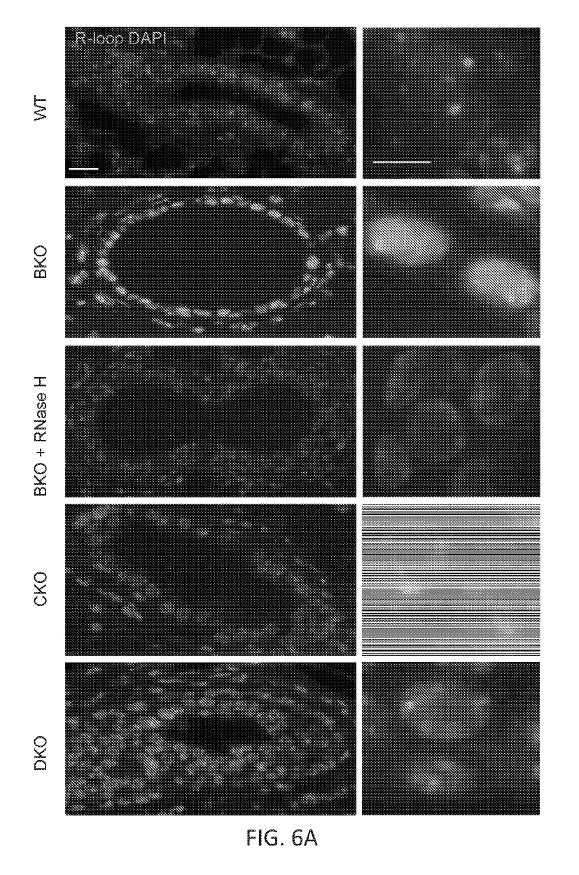

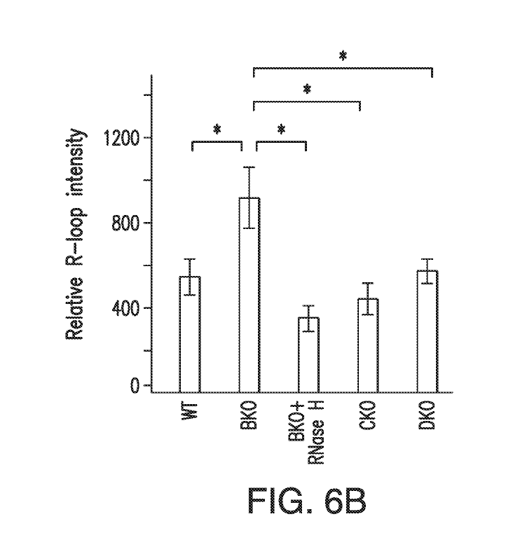

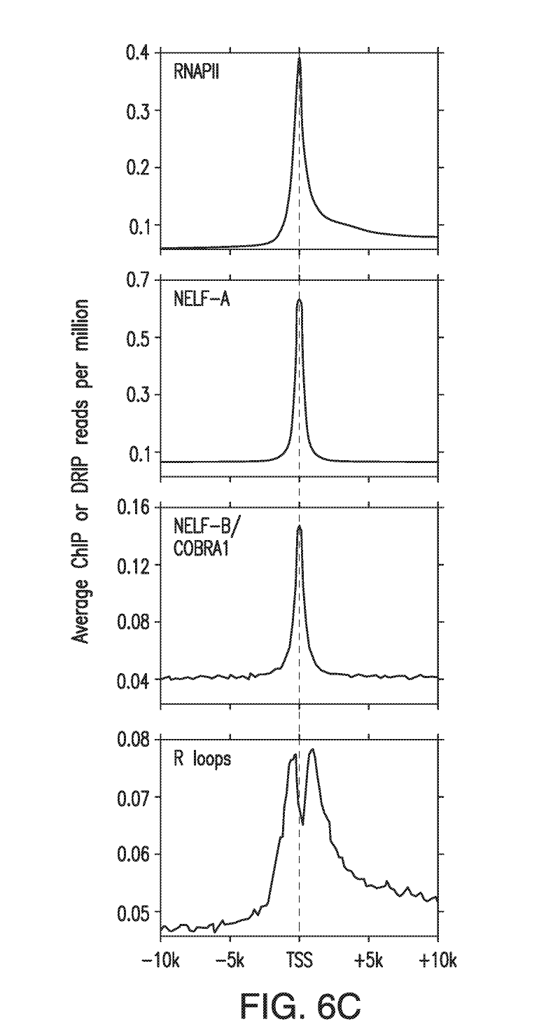

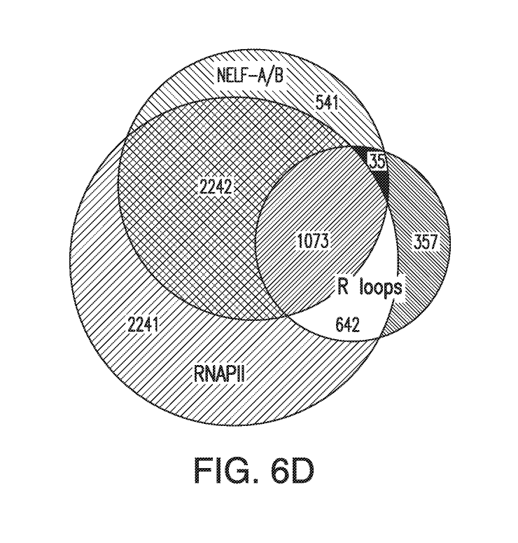

Given the well-documented function of COBRA1 in transcriptional elongation and the recently reported link between BRCA1 and R-loop accumulation, it was investigated whether the functional antagonism between Cobra1 and Brca1 in mammary tumorigenesis was associated with R-loop dynamics. Luminal epithelial cells from BKO exhibited more pronounced pan-nuclear staining with an R-loop-specific antibody versus age-matched WT mice (FIG. 6a,b), consistent with the in vitro findings of elevated R-loop accumulation upon BRCA1 knockdown. As a critical control, the R-loop signal in BKO mammary epithelium was obliterated by pre-treatment of the fixed tissue samples with RNase H, a nuclease that specifically degrades RNA in the R-loop structure (FIG. 6a,b). Remarkably, the R-loop intensity in DKO mammary epithelial cells was significantly lower than that in BKO (FIG. 6a,b). Thus, a COBRA1-dependent event, likely its well-characterized role in RNAPII pausing, can contribute to R-loop accumulation.

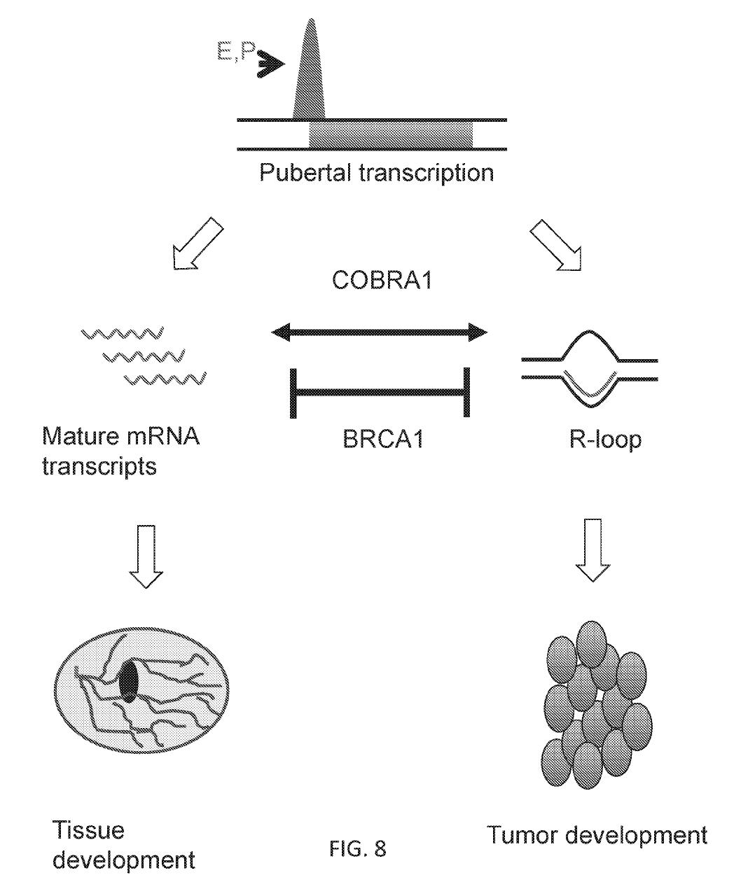

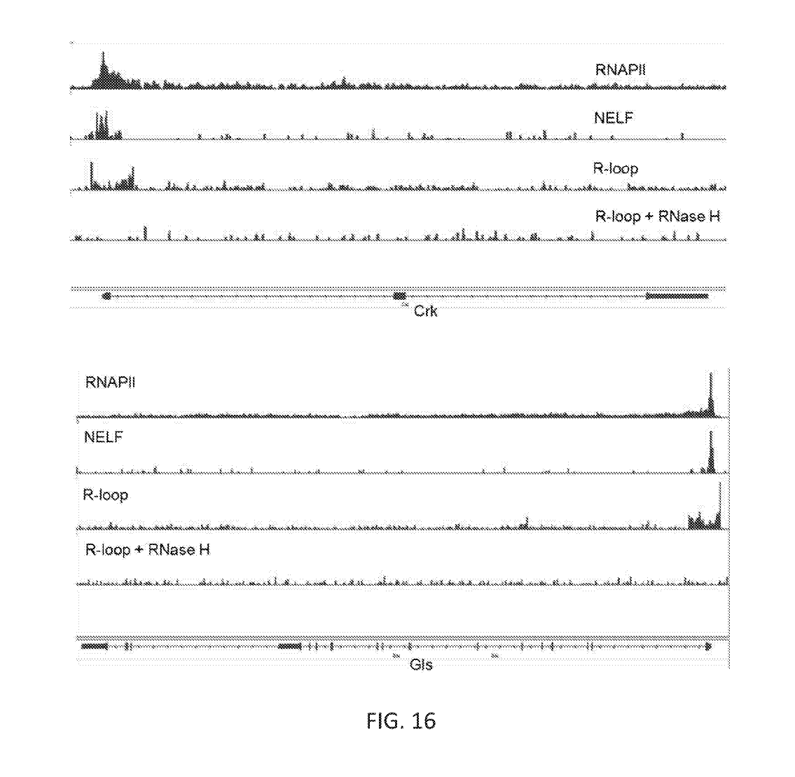

To validate the association between COBRA1, RNAPII pausing, and R-loops in mammary epithelium, primary epithelial cells were isolated from mouse mammary tissue and chromatin immunoprecipitation-sequencing (ChIP-seq) and DNA-RNA immunoprecipitation-sequencing (DRIP-seq) was performed. ChIP-seq of RNAPII and the NELF complex (NELF-A and NELF-B/COBRA1) indicated that both signals were enriched at TSS (FIG. 6c, FIG. 16), consistent with the known RNAPII-pausing function of NELF. As detected by DRIP-seq, average R-loop signals in the same mammary epithelial cells exhibited a TSS-enriched pattern (FIG. 6c). Notably, there was a marked overlap between the genes with TSS-enrichment of NELF, RNAPII, and R loops (P=0, FIG. 8d and FIG. 16). Taken together, the genomic data indicate that COBRA1-mediated RNAPII pausing contributes to R-loop dynamics in mammary epithelium.

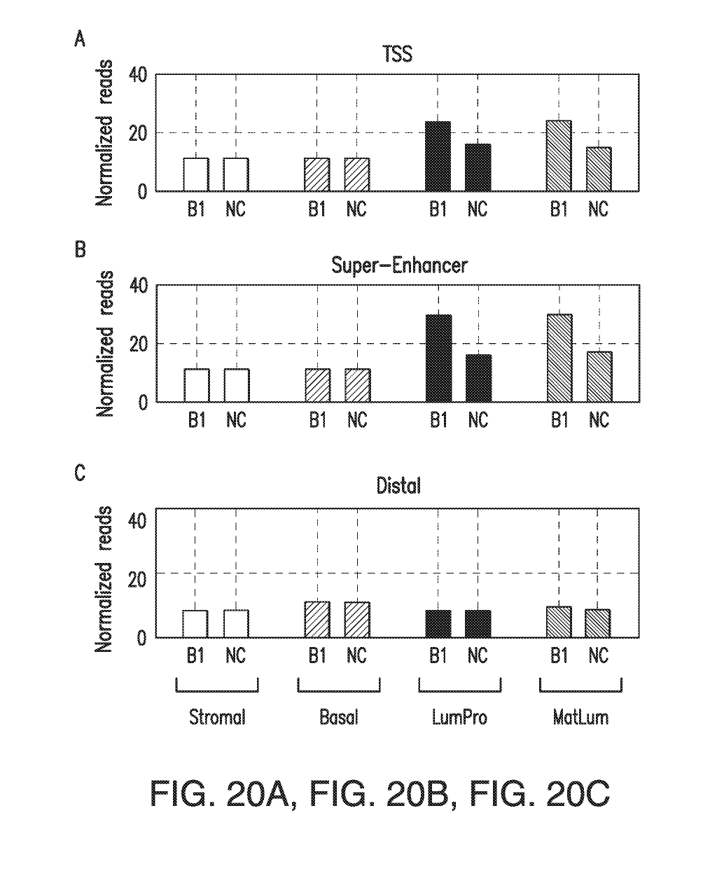

Following R-loop-specific immunostaining of formalin fixed paraffin-embedded (FFPE) cancer-free breast tissue of BRCA1 mutation-carrying women, next-generation sequencing was used to identify the specific genomic locations of BRCA1 mutation-associated R-loop accumulation. Specifically, fresh breast tissue samples from BRCA1 mutation carriers and non-carriers were subjected to fluorescence-activated cell sorting (FACS) using established cell surface markers (EpCAM and CD49f)1,2. Each clinical sample was sorted to four populations: stromal cells, basal epithelial cells, luminal progenitor cells (LumPro), and mature luminal epithelial cells (MatLum). The R-loop-specific antibody was used in DNA-RNA immunoprecipitation-sequencing (DRIP-seq)3. An average of 50 million mapped reads were obtained per DRIP-seq reaction. Consistent with the immunostaining data, R-loop accumulation in BRCA1 mutant carriers (B1) was only observed in the luminal progenitor (yellow) and mature luminal cells (red), not stromal (blue) or basal epithelial (green) cells (FIG. 20A-B). Furthermore, BRCA1 mutation-associated R-loop accumulation occurs most notably at transcription start sites (TSS, FIG. 20A) and luminal super-enhancers (FIG. 20B), which are known to drive gene expression for cell fate determination4-6. In contrast, genomic regions distal to both enhancers and genic regions did not exhibit any appreciable difference between carriers and non-carriers (FIG. 20C). Gene ontology indicates that BRCA1 mutation-associated R-loop accumulates preferentially at gene loci encoding luminal differentiation-related transcription factors (e.g., ERα, GATA3, ELF5, and ZNF217) and known luminal markers (e.g., ALDH1A37), thus further supporting a role of BRCA1 in luminal lineage-specific transcriptional regulation.

v. Normal Luminal Epithelial Cells from Cancer-Free BRCA1 Mutation Carriers Exhibit R-Loop Accumulation Due to BRCA1 Haploinsufficiency

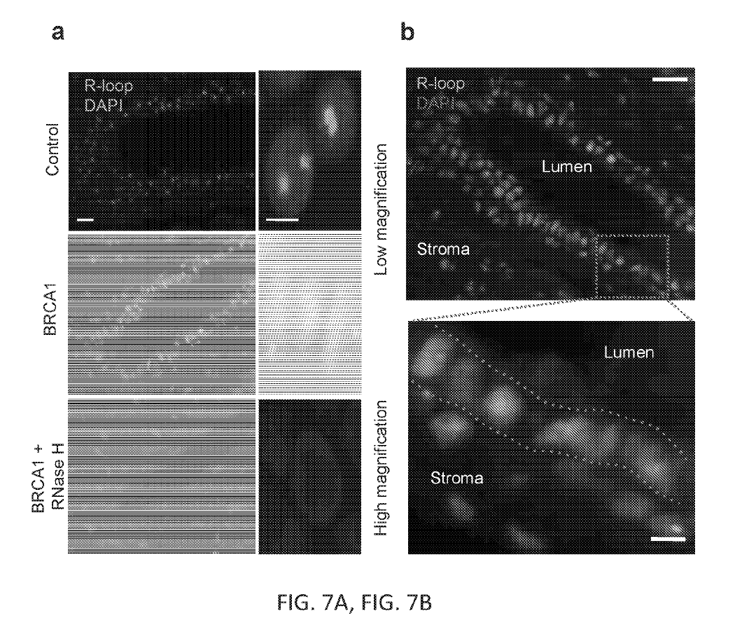

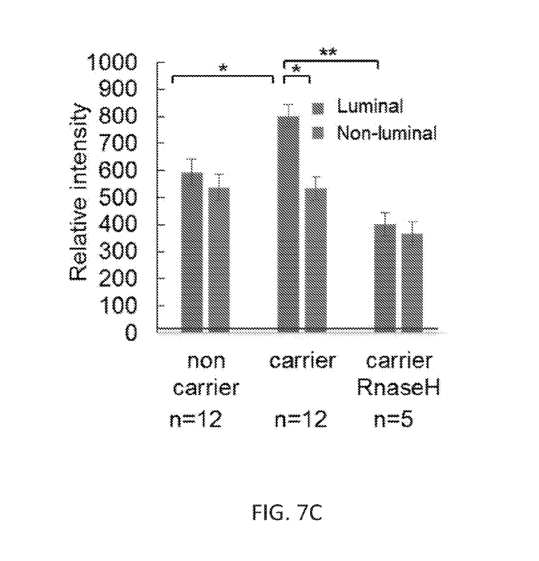

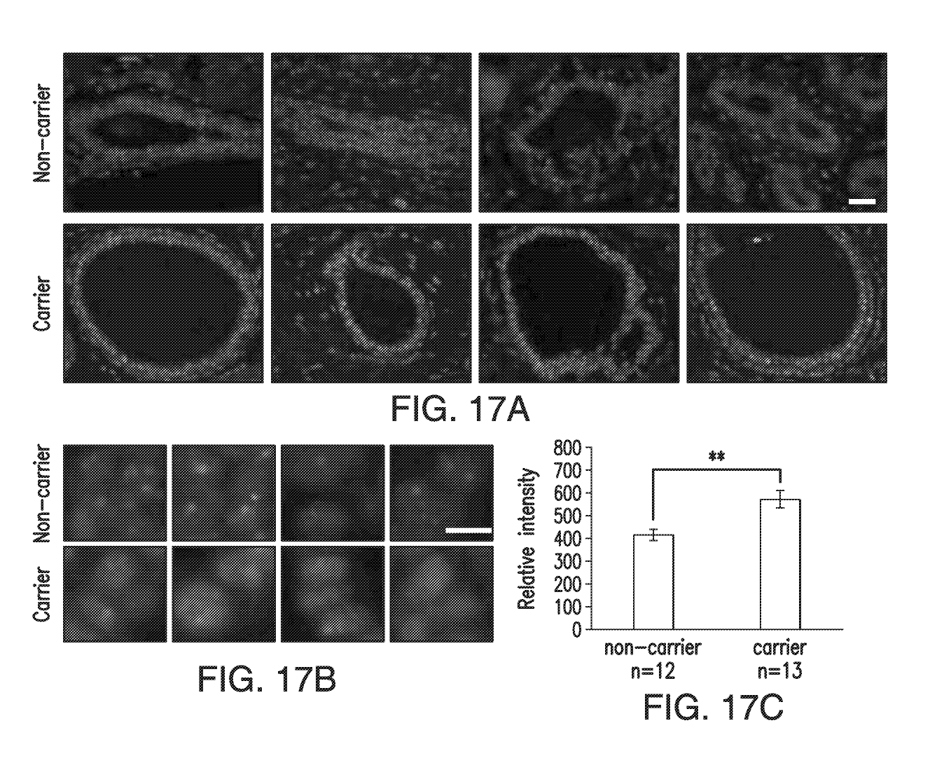

To extend the findings from these animal models, R-loop signals were examined in normal breast tissue of cancer-free BRCA1 mutation-carrying women. Breast epithelial cells from the non-carrier group displayed relatively low pan-nuclear staining, with one or two distinct nuclear foci per cell (FIG. 7a). In contrast, breast epithelial cells from the BRCA1 mutation carriers had pronounced pan-nuclear R-loop staining that was sensitive to RNase H (FIG. 7a). Furthermore, R-loop accumulation occurred preferentially in the luminal epithelial cells of the BRCA1 mutation carrier group (FIG. 7b,c). In comparison, non-luminal cells (basal epithelial and stromal) from the same carriers did not exhibit elevated R-loop signals compared to their counterparts in the control group (FIG. 7b,c). This finding was confirmed using a separate cohort of BRCA1 mutation carriers (FIG. 17). Thus, R-loop accumulation represents an early sign of BRCA1 haploinsufficiency in BRCA1 mutation carriers, prior to breast cancer development.

2. Discussion

The universality of the extensively characterized DSB repair activity of BRCA1 stands in stark contrast to its sex and tissue-specific tumor suppressor function. By using mammary gland-specific KO mouse models and focusing on a critical stage of hormone-driven mammary gland development, a DSB repair-independent functional interplay between BRCA1 and a key regulator of transcriptional elongation during normal tissue development and tumorigenesis was identified. These findings underscore the importance of studying physiologically relevant tissue context to elucidate early molecular events that drive initiation of tissue-specific BRCA1-associated tumors.

BRCA1-associated tumorigenesis has been linked with various actions of ovarian hormones. Antagonism between BRCA1 and COBRA1 strikes a critical balance between promotion of ovarian hormone-driven mammary gland development and prevention of breast cancer (FIG. 8). In this model, transcription in response to surging ovarian hormones leads to production of developmentally important gene products that are obligatory to ductal morphogenesis. On the flip side of the same coin, activation of the transcription program also yields a less desirable by-product in the form of R loops, the accumulation of which can cause aberrant gene expression, epigenetic changes, and genome instability. In this regard, R-loops are analogous to spontaneous mutations resulting from DNA replication in dividing adult stem cells, which was indicated to be the underlying cause for many cancer types. These two outcomes of the same transcriptional event are controlled by COBRA1 and BRCA1 in an antagonistic manner. COBRA1 ensures proper ductal transcription and development by counteracting the BRCA1 effect on these events. Reciprocally, BRCA1 attenuates COBRA1-dependent R-loop accumulation in mammary epithelium. Thus, this model explains why loss of COBRA1 manifests the BRCA1-mediated inhibition of ductal morphogenesis as observed in CKO, and conversely, why BKO mice without functional BRCA1 suffer from accumulated R loops as a result of unopposed COBRA1 actions. DKO animals with simultaneous loss of BRCA1 and COBRA1 are able to reestablish a quasi-balanced state, in which a partially restored transcription program is potent enough to drive normal tissue development yet sufficiently tamed to avoid accumulation of R loops.

It is counterintuitive that DKO mice have reduced tumorigenesis compared to BKO, yet exhibit an expanded luminal epithelial compartment. One explanation is that precocious differentiation in the luminal compartment could result in exhaustion of the progenitor cell pool that would otherwise accumulate, with a high propensity to develop BRCA1-associated tumors. In other words, the multilayer phenotype in DKO mammary glands could reflect a “self-cleansing” mechanism for eliminating the cell of origin for BRCA1-associated tumors.

The DSB repair-independent interaction between BRCA1 and COBRA1 in tissue development and tumorigenesis represents a conceptual departure from the prevailing DNA repair-centric paradigm for BRCA1 biology. However, it is important to note that this work does not refute the DNA repair function of BRCA1 as an integral component of its tumor suppressor activity. In fact, the residual tumor incidence in DKO compared to WT can be due to persistent DNA repair deficiency in the absence of BRCA1. The functional antagonism between BRCA1 and COBRA1 is distinct from the previously reported interplay between BRCA1 and 53BP182,83, which is attributed to the competition between HR and non-homologous end-joining pathways of DSB repair. In contrast, BRCA1/COBRA1 antagonism is clearly DSB-independent. Furthermore, while 53BP1 deletion rescued developmental defects in Brca1-deficient embryos, the genetic complementation between Brca1 and Cobra1 is apparently more tissue-restricted. The universal function of BRCA1 in DNA repair and its tissue-dependent crosstalk with COBRA1 in transcription are both required for maximal suppression of tumorigenesis in the unique hormonal milieu of breast epithelium.

Based on evidence from various in vitro studies, collision between R loops and DNA replication forks can be the primary source of R-loop-associated DSB. However, unlike monotypic cancer cell lines cultured in vitro, normal human and mouse ER+ luminal epithelial cells in vivo are predominantly non-proliferative. Rather, in response to ovarian hormones, a paracrine action of these ER+ cells stimulates proliferation of their neighboring ER− epithelial cells. This important idiosyncrasy of normal breast epithelium in vivo likely adds another level of complexity to the functional consequences of R-loop accumulation, which could differ between proliferating and non-proliferating breast epithelial cells. R-loop accumulation was observed across the entire BRCA1-deficient luminal epithelial compartment, and it did not correlate with the DSB marker γH2AX. Therefore, in addition to DSBs resulting from R-loop collision with replication forks in proliferating epithelial cells, other known effects of R-loop accumulation including DSB-independent genetic instability, epigenetic alterations, and gene expression changes can also contribute to tumorigenesis in breast epithelium.

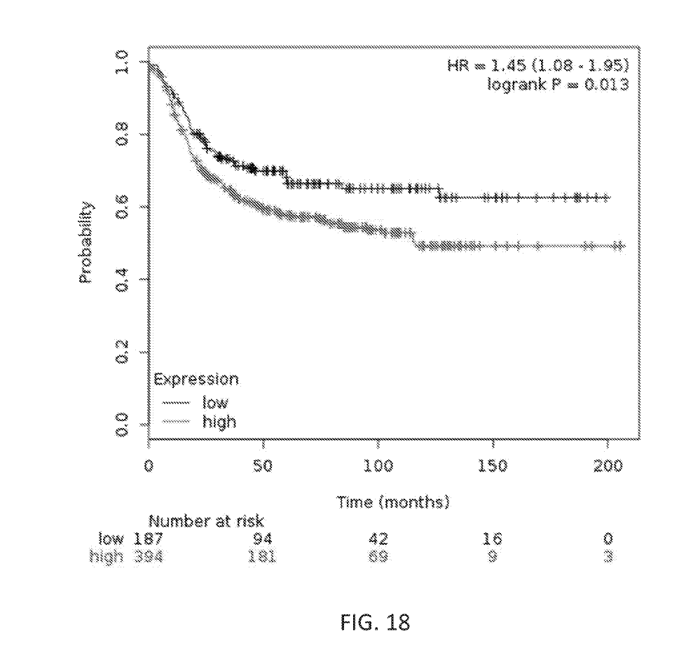

Considering that all non-cancerous somatic cells of a BRCA1 mutation carrier have one WT and one mutant BRCA1 allele, it is intriguing that elevated R-loop signals were predominantly observed in luminal epithelial cells in a BRCA1-haploinsufficient manner. Cell type-specific R-loop accumulation can represent an early hallmark in BRCA1-associated tumorigenesis, which can be used as a risk-assessing tool for BRCA1 mutation carriers, especially those BRCA1 mutations with equivocal disease association. It is also worth noting that, when compared with dissected normal breast epithelium from cancer-free individuals, tumor samples from triple negative breast cancer patients have significantly elevated COBRA1 mRNA (2.55 fold, P=4.83×10⁻⁶). In addition, high COBRA1 expression is associated with poor outcome for patients with basal-like breast cancer (FIG. 18). As most BRCA1-associated breast tumors fall into the triple-negative and basal-like types, the previously unappreciated role of COBRA1 in R-loop accumulation and luminal progenitor cell expansion offers a potential DSB repair-independent target for reducing BRCA1-associated cancer risk.

3. Methods

Mice: Cobra/Nelf-bf/f mice have been described previously61. WTV-Cre,Cobraf/f mice were generated by breeding MMTV-Cre line A animals with Cobraf/f mice. Trp53f/f (Trp53tm1Brn), Ink4-Arf KO, and Brca1f/f mice were obtained from Mouse Model of Human Cancer Consortium (MMHCC), National Cancer Institute. EIIa-Cre was purchased from the Jackson Laboratory, and used to generate the whole-body hemizygous deletion strain Brca1+/−,Cobra+/− per previously described procedures. The strains were in a mixed genetic background.

Breast tissue cohorts: Cancer-free breast tissue was procured from women either undergoing cosmetic reduction mammoplasty, diagnostic biopsies, or mastectomy. All donors signed a written consent form authorizing the use of the specimens for breast cancer-related laboratory investigations.

Whole mount analysis of the mammary glands: Inguinal mammary glands from mice of different age groups as indicated were used for whole mount staining. The inguinal fat pads were gently isolated and spread onto a glass slide. The glands were fixed in Carnoy's fixative (ethanol: chloroform: glacial acetic acid, 60:30:10) overnight at room temperature. The glands were rehydrated in descending grades of alcohol (70%, 50%, 30%) for 15 min each, then washed with distilled water prior to overnight staining in Carmine alum (1 g carmine, 2.5 g aluminum potassium sulfate boiled for 20 min in distilled water, filtered and brought to a final volume of 500 ml). The stained glands were dehydrated in ascending grades of alcohol (70%, 70%, 90%, 95%, 100%, 100%) for 15 min each, and cleared with Citrisolv reagent (Fisher, Cat#. 22-143975). Samples were and examined under a Nikon SMZ1000 dissection microscope. Duct length was measured from calibrated images using Eclipse software. Average length of three longest ducts from nipple region was taken as the ductal length of each animal.

Immunohistochemistry (IHC) and immunofluorescence staining: Primary antibodies used were anti-NELF-B/COBRA161, anti-milk protein (Nordic Immunology, RAM/MSP), anti-R-loop (S9.6; Karafast, ENH001), anti-BrdU (GE Healthcare, RPN20), anti-□H2AX (Cell Signaling, 9718), anti-K8 (Developmental Studies Hybridoma Bank, TROMA-1), anti-K14 (Covance, PRB-155P), anti-Rad51 (Santa Cruz, sc-8349), and anti-ERα (Santa Cruz, sc-542).

Mammary glands were fixed in 10% Neutral buffered formalin for 18 hr at 40 C and paraffin embedded. Sections of 2 or 3 μM in thickness were used for hematoxylin-eosin (H&E) staining and IHC. Samples were baked at 700 C for 15 min, then de-paraffinized by three 5-min extractions in 100% xylene, followed by 3-min each of descending grade of alcohol (100%, 95%, 70%, 50%). Samples were washed briefly with PBS before transferring to boiling antigen-unmasking solution (Vector Labs, H-3300) for 20 min. For IHC, sections were pre-treated with 3% hydrogen peroxide for 10 min before blocking. Blocking was performed with 10% normal goat serum in PBS for 1 hr at room temperature followed by primary antibody incubation overnight at 40 C. For detection with primary antibody using the immune enzymatic method, the ABC peroxidase detection system (Vector Labs, PK-6105) was used with 3,3′-diaminobenzidine (DAB) as substrate (Vector Labs, SK-4105) according to manufacturer's instruction.

For immunofluorescence staining, sections were incubated with Alexa-488 and Alexa-546-conjugated secondary antibodies (Life Technologies), mounted with Vectashield mounting medium with DAPI (Vector Labs, H-1200), and examined with an Olympus FV1000 confocal microscope or Nikon Eclipse Ni fluorescent microscope. For BrdU pulse-labeling, mice were intraperitoneally injected with cell proliferation labeling reagent (GE Healthcare, RPN201) at 16.7 ml/kg. For BrdU/Rad51 and BrdU/γH2AX double staining, mice were first injected with BrdU and then X-rayed at 20 Gy using a Faxitron cabinet X-ray system (Model 43855F). Mammary glands were harvested 3 hr after labeling.

R-loop immunofluorescence staining and intensity quantification: After de-paraffin and re-hydration, samples were treated in boiling antigen-unmasking solution (Vector Labs, H-3300) for 1 hr. After antigen unmasking, samples were cooled down to room temperature and then treated with 0.2×SSC buffer (Ambion, AM9763) for 20 min with gentle shaking. Samples were then incubated overnight in primary antibody S9.6 (S9.6; Karafast, ENH001) at 1:100 dilution in PBS containing 1% normal goat serum and 0.5% Tween-20 at 37° C. The next day, samples were washed with PBS containing 0.5% Tween-20 for 5 min three times. Samples were incubated with Alexa-488-conjugated secondary antibody (Life Technologies) at 1:1000 dilution in PBS containing 1% normal goat serum and 0.5% Tween-20 at 37° C. for 2 hr. Samples were washed twice with PBS containing 0.5% Tween-20 for 3 min followed by 3 min PBS wash twice. Samples were then mounted with Vectashield mounting medium with 4′,6-diamidino-2-phenylindole (DAPI, Vector Labs, H-1200), and examined under a Nikon Eclipse Ni fluorescent microscope. For samples pre-treated with RNase H, an overnight treatment of RNase H (NEB, M0297S) is carried out after 0.2×SSC treatment. Samples were then washed in PBS for 5 min three times before incubation with the primary antibody.

R-loop intensity was determined using MetaMorph Microscopy Automation and Image Analysis Software 7.8. At least four images, each of which contained a minimum of one complete epithelial duct, were acquired for each sample. For each image, the DAPI signal was used to create a mask of the nucleus in either the luminal epithelial compartment or the basal/stromal compartments. The R-loop intensity was determined by calculating the average intensity in the mask. The final R-loop intensity for each sample is the average of all images.

Statistics: All data are expressed as means±s.e.m. Differences between two groups were compared using a two-tailed unpaired Student's t test. P<0.05 was considered statistically significant. For mouse tumor studies, log-rank test in the GraphPad Prism software was used.

Primary mammary epithelial cell (MEC) isolation and flow cytometry: Thoracic and inguinal mammary glands from virgin mice were isolated in sterile condition and lymph nodes from inguinal gland were removed. Single cells were prepared using published protocol97 with minor modifications. All reagents were purchased from StemCell Technologies (Vancouver, Canada), unless otherwise indicated. Briefly, the isolated glands were minced using scissors and digested for 15-18 hr at 370 C in DMEM F-12 (Cat#36254) containing 2% FBS, Insulin (5 mg/ml), Penicillin-Streptomycin and a final concentration of 1 mg/mL Collagenase and 100 U/ml Hyaluronidase (Cat#07919). After vortexing, epithelial organoids were collected by centrifugation at 600 g for 4 min. Red blood cells (RBCs) in the resulting pellets were lysed with 0.8% NH4Cl. The epithelial organoids were then digested by pipetting with 2 ml of 0.05% pre-warmed Trypsin (Life Technologies, 25300) for 2 min, followed by washing in ice-cold Hanks Balanced Salt Solution (Cat#37150) with 2% FBS (HF). The cells were resuspended in 5 mg/ml Dispase (Cat#07913) with 0.1 mg/ml DNAse I (Sigma-Aldrich, D4513). After trituration for 1-2 min. the cells were resuspended in ice-cold HF, and single cells were prepared by filtering the cell suspension through a 40-μm cell strainer (Fisher, Cat#22363547). Cells were counted and resuspended in HF at a concentration of 1×106 cells/100 μL. Cell were incubated for 10 min on ice with 10% rat serum (Jackson Laboratories, 012-000-120) and Fc receptor antibody (BD Biosciences, 553141). After blocking, cells were incubated for 20 min with antibodies for the following cell-surface markers: Ep-CAM-PE (BioLegend, 118206), CD49f-FITC (BD Biosciences, 555735), CD31-Biotin (BD Bioscience, 553371), CD45 biotin (BioLegend, 103103), TER-119 Biotin (BioLegend, 103511), and CD49b-Alexa Fluor 647 (BioLegend, 104317) followed by Streptavidin-Pacific Blue (Invitrogen, S11222). 7-AAD (BD Biosciences, 559925) was added 10 min before analysis. CD49b+ cells were gated using a fluorescent-minus-one (FMO) control, in which all antibodies except CD49b-Alexa 647 were used. Sorting was performed with a Moflo Astrios cell sorter (Beckmen Coulter). Data were analyzed using FACSDiva software. Purity of the stromal, luminal, and myoepithelial populations were verified by real-time PCR analysis of Vimentin (stromal), Keratin-18 (luminal), Keratin 5 (myoepithelial), and Keratin-14 (myoepithelial) mRNA.

Gene expression profiling: Triplicates of RNA samples from different mice of each genotype were labeled using the Illumina® TotalPrep™ RNA amplification kit (Ambion, Cat. #AMIL1791) and subsequently hybridized to Illumina mouse whole genome gene expression BeadChips (MouseRef-8 version 2.0, Illumina). BeadChips were scanned on an iScan Reader (Illumina) using iScan software (version 3.3.29, Illumina). For further analysis, the scanned data were uploaded into GenomeStudio® software (version 1.9.0, Illumina) via the gene expression module (Direct Hyb).

Bioinformatics analysis of microarray data: For each of the time points, genes were identified that are affected by Cobra1 KO (CKO-affected) and those that are eventually rescued by double KO (DKO-rescued). CKO-affected genes are defined as the genes that show ≥2.0 fold enrichment (either up or down) in CKO mice compared to corresponding WT control mice, with P≤0.05. DKO-rescued genes are defined as those CKO-affected genes that had either (1) ≤1.5 fold enrichment (either up or down, P<0.05) in DKO versus WT control mice, or (2) fold of changes in DKO versus WT (P<0.05) no more than 50% of those in CKO versus WT, or (3) any fold of changes in DKO versus WT with P value larger than 0.05. Table 3 shows the total number of CKO-affected and DKO-rescued genes for the indicated time points.

Pubertal, estrogen and progesterone signature genes were extracted from previously published studies to identify the overlap with CKO-affected or DKO-rescued genes. Table 3 shows the overlap among CKO-affected/DKO-rescued genes with pubertal, estrogen and progesterone genes. The statistical significance (p-value) of the overlap was calculated using the Fisher's exact test:

$$p = 1 - {\sum }_{t = 0}^{c - 1} C ( m , t ) C ( N - m , n - t ) / C ( N , {n}^{′} )$$

where N is the total number of genes in the experiment; m,n is the selected affected/rescued and previously published signature genes respectively and o is the overlap among those genes. C(n,k) is the bionomial coefficient.

In vitro HR-based DSB repair assay: The homology directed repair (HDR) assay was performed using established methods. The recombination substrate, pDR-GFP, contains two inactive GFP genes, one of which is due to the presence of an I-SceI endonuclease recognition sequence. This DNA is integrated into a single site in HeLa cells. On day 1, siRNAs specific for a control sequence, COBRA1, and BRCA1 were transfected, using Oligofectamine (Invitrogen), into wells containing HeLa-DR-GFP cells. On day 3, the cells were re-transfected with the same siRNAs plus a plasmid for the expression of the I-SceI endonuclease using the Lipofectamine 2000 transfection reagent (Invitrogen). On day 6, cells were released from the monolayer using trypsin and the fraction of GFP+ cells was determined using a FACS-Calibur analytical flow cytometry instrument. Results were normalized to the percent of GFP+ cells in the sample in which the control siRNA was transfected and plotted ±s.e.m. Assays were performed in triplicate and the significance of the results was analyzed using the two-tailed student's t-test.

Chromatin Immunoprecipitation (ChIP) assay: Primary mammary epithelial cells were isolated as described above, with the following modification for the tissue digestion step. Briefly, thoracic and inguinal mammary glands were isolated from 6-8 week virgin mice. Lymph nodes were removed from the inguinal glands. Tissues were quickly minced with scissors and digested in DMEM F-12 containing 2% FBS, Penicillin-Streptomycin and a final concentration of 300 U/ml Collagenase and 100 U/ml Hyaluronidase (StemCell Technologies, Cat#07912) for 45 min with gentle shaking. Samples were vortexed vigorously for 15 sec every 15 min during the digestion. After tissue digestion mammary organoids were collected and RBCs were lysed. Organoids were further digested by Trypsin and Dispase, and single mammary epithelial cells were obtained after passing through cell strainer, as described above. Cells were crosslinked in crosslinking solution (1% formaldehyde, 10% FBS in PBS) for 10 min at room temperature, and the reaction was terminated with 125 mM Glycine for 5 min at room temperature. The crosslinking reagents were removed by spinning at 1600 g for 5 min at 40 C, and cells were washed with cold PBS containing 2% FBS twice at 1600 g for 5 min. From this step on until ChIP elution, all buffers were prepared with freshly added cocktail of phosphatase and protease inhibitors (10 mM sodium fluoride, 10 mM sodium pyrophosphate tetrabasic, 2 mM sodium orthovanadate, 1 μg/ml leupeptin, 1 μg/ml aprotinin, 1 μg/ml pepstatin and 1 mM PMSF). Cells were lysed on ice for 10 min using lysis buffer (5 mM HEPES, pH 7.9, 85 mM KCl, 0.5% Triton-X-100). Supernatant was removed after spinning at 1600 g for 5 min at 40 C, and cells were resuspended for 10 min at 40 C in nuclei lysis buffer [50 mM Tris-HCl, pH 8.0, 10 mM EDTA, 0.5% (wt/vol) SDS]. Nuclei were isolated by spinning at 14,000 g for 10 min at 40 C and resuspended in RIPA buffer [10 mM Tris-HCl, pH 7.5, 140 mM NaCl, 1 mM EDTA, 0.5 mM EGTA, 1% Triton-X-100, 0.1% (wt/vol) SDS, 0.1% sodium deoxycholate]. Chromosomal DNA was sonicated using a probe sonicator 30 s on and 30 s off (4 cycles) at 25% power on ice. Cells were centrifuged at 14,000 g for 10 min and the supernatant was saved. Protein-A Dynabeads were washed and prebound with antibodies (anti-RNAPII, Abcam, ab5408, anti-NELF-A/B) for 2 hr at 40 C. Sonicated DNA and antibody-bound Dynabeads were incubated at 40 C overnight. For RNAPII ChIP, Dynabeads were washed 3 times in RIPA buffer and once in TE buffer (10 mM Tris-HCl, pH 8.0, 10 mM EDTA), then reverse-crosslinked and eluted. For NELF-A/B ChIP, Dynabeads were washed twice in TE Sarcosyl buffer (50 mM Tris-HCl pH 8.0, 2 mM EDTA, 0.2% sarcosyl), twice in TSE1 buffer [150 mM sodium chloride, 20 mM Tris-HCl pH 8.0, 2 mM EDTA, 0.1% (wt/vol) SDS, 1% Triton-X-100], twice in TSE2 buffer [500 mM sodium chloride, 20 mM Tris-HCl, pH 8.0, 2 mM EDTA, 0.1% (wt/vol) SDS, 0.1% Triton-X-100], twice in TSE3 buffer (250 mM lithium chloride, 10 mM Tris-HCl, pH 8.0, 1 mM EDTA, 1% sodium deoxycholate, 1% NP-40), and twice in TE buffer. Samples were subsequently reverse-crosslinked and eluted.

DNA-RNA ImmunoPrecipitation (DRIP): DRIP assay was performed following the established protocol47. Briefly, primary mammary epithelial cells were isolated as described above in the ChIP assay. Cells were washed twice in PBS, and resuspended in TE (Sigma, T9285) containing a final concentration of 0.5% SDS and proteinase K (Roche, 03115828001). Samples were incubated overnight at 370 C. Genomic DNA was extracted using phenol/chloroform (Sigma, P2069) in phase lock tubes (SPRIME, 2302840) and ethanol precipitated. DNA was digested using established restriction enzyme cocktail (HindIII, EcoRI, BsrGI, XbaI and SspI) overnight at 370 C. Digested DNA was cleaned up by phenol-chloroform extraction and ethanol precipitation. For DRIP, digested DNA was incubated with S9.6 antibody overnight at 40 C in binding buffer (10 mM sodium phosphate, 140 mM sodium chloride, 0.05% Triton X-100 in TE). RNase H-treated sample was used as a negative control for DRIP. Dynabeads were added the next day for 2 hr. Bound Dynabeads were then washed with binding buffer three times at room temperature. DNA was eluted, phenol-chloroform extracted, and ethanol precipitated. DRIP DNA was sonicated using Covaris (Model S220) before library preparation.

Library preparation and sequencing: ChIP-seq and DRIP-seq libraries were built following the instruction of MicroPlex library preparation kit (Diagenode, C05010011). For RNAPII ChIP-seq, 1 ng of ChIP DNA was used for a total of 15 cycles of PCR amplification. For NELF-A/B ChIP-seq, 0.2 ng ChIP DNA were used for a total of 18 cycles of PCR amplification. For DRIP-seq, a total of 15 cycles of PCR amplification was performed. After amplification, libraries were purified using Agencourt AMPure XP system (Beckman Coulter, A63880) following the product manual. Quantity of the libraries was measured with Qubit dsDNA HS Assay Kit (Life Technologies, Q32851), and quality of the libraries was verified using Bioanalyzer 2100. Libraries were pooled based on index sequences. 14 pM library pool was loaded to Illumina HiSeq2000 and sequenced by 50 bp single-read sequencing module. After sequencing run, demultiplexing with CASAVA was employed to generate the FASTQ file for each sample. Two biological replicates were used for RNAPII ChIP-seq and DRIP-seq and between 38-64 million total reads were obtained for each biological sample.

Bioinformatics analysis of ChIP-seq and DRIP-seq: Reads in FASTQ file were aligned to mouse genome by BWA99, a software package for mapping low-divergent sequences against reference genome, and only unique mapped reads were selected for analysis. BELT100, a peak-calling program, was used to identify the peaks (binding sites) for uniquely mapped reads. In brief, BELT employs a bin-based enrichment threshold to define peaks and applies a statistical method to control false discovery rate (FDR). With different parameters, BELT identifies different number of peaks, and generally higher number of peaks is more likely to be associated with higher FDR. In this study, using the same parameters, the estimated FDR of identified peaks for all samples are all less than 8% except for NELF-B ChIP-seq. TSS-bound peaks were identified by 1 bp overlap to TSS upstream/downstream 1 kb region of mouse reference genes. Venn diagrams of the overlap genes were generated by BioVenn101, a web application for comparison and visualization of biological lists. The p-value of the significance of the overlap in the Venn diagrams was calculated by hypergeometric distribution.

Primer sequences: For RT-PCR:
(SEQ ID NO: 1)

| | |
|---|---|
| 18sRNA-F: | 5′-GAATTCCCAGTAAGTGCGGG-3′, |

(SEQ ID NO: 2)

| | |
|---|---|
| 18sRNA-R: | 5′-GGGCAGGGACTTAATCAACG-3′. |

(SEQ ID NO: 3)

| | |
|---|---|
| Cobra1-F: | 5′-ACAACTTCTTCAGCCCTTCCC-3′, |

(SEQ ID NO: 4)

| | |
|---|---|
| Cobra1-R: | 5′-TCTGCACCACCTCTCCTTGG-3′. |

(SEQ ID NO: 5)

| | |
|---|---|
| Brca1-F: | 5′-AGCAAACAGCCTGGCATAGC-3′, |

(SEQ ID NO: 6)

| | |
|---|---|
| Brca1-R: | 5′-ACTTGCAGCCCATCTGCTCT-3′. |

(SEQ ID NO: 7)

| | |
|---|---|
| p16Ink4a-F: | 5′-GAACTCTTTCGGTCGTACCCC-3′, |

(SEQ ID NO: 8)

| | |
|---|---|
| p16Ink4a-R: | 5′-CGTGAACGTTGCCCATCAT-3′. |

(SEQ ID NO: 9)

| | |
|---|---|
| p19Arf-F: | 5′-CTTGAGAAGAGGGCCGCAC-3′, |

(SEQ ID NO: 10)

| | |
|---|---|
| p19Arf-R: | 5′-AACGTTGCCCATCATCATCA-3′. |

(SEQ ID NO: 11)

| | |
|---|---|
| p53-F: | 5′-GAGACAGCAGGGCTCACTCC-3′, |

(SEQ ID NO: 12)

| | |
|---|---|
| p53-R: | 5′-TGGCCCTTCTTGGTCTTCAG-3′. |

(SEQ ID NO: 13)

| | |
|---|---|
| Ctse-F: | 5′-ATTGGCAGATTGCCCTGGAT-3′, |

(SEQ ID NO: 14)

| | |
|---|---|
| Ctse-R: | 5′-GCCTTCGGAGCAGAACATCA-3′. |

(SEQ ID NO: 15)

| | |
|---|---|
| Prom2-F: | 5′-TGACCTGGATAAGCACCTGG-3′, |

(SEQ ID NO: 16)

| | |
|---|---|
| Prom2-R: | 5′-AAGCTCTGAAGCTCCTGCTG-3′. |

(SEQ ID NO: 17)

| | |
|---|---|
| Acot1-F: | 5′-ATGGCAGCAGCTCCAGACTT-3′, |

(SEQ ID NO: 18)

| | |
|---|---|
| Acot1-R: | 5′-CCCAACCTCCAAACCATCAT-3′. |

(SEQ ID NO: 19)

| | |
|---|---|
| Ramp2-F: | 5′-GCCTCATCCCGTTCCTTGTT-3′, |

(SEQ ID NO: 20)

| | |
|---|---|
| Ramp2-R: | 5′-CCTGGGCATCGCTGTCTTTA-3′. |

(SEQ ID NO: 21)

| | |
|---|---|
| Vwf-F: | 5′-CGACCTGGAGTGTATGAGCC-3′, |

(SEQ ID NO: 22)

| | |
|---|---|
| Vwf-R: | 5′-ACACACTTGTTTTCGTGCCG-3′. |

(SEQ ID NO: 23)

| | |
|---|---|
| Gata3-F: | 5′-GATGTAAGTCGAGGCCCAAG-3′, |

(SEQ ID NO: 24)

| | |
|---|---|
| Gata3-R: | 5′-GCAGGCATTGCAAAGGTAGT-3′. |

(SEQ ID NO: 25)

| | |
|---|---|
| K18-F: | 5′-ACTCCGCAAGGTGGTAGATGA-3′, |

(SEQ ID NO: 26)

| | |
|---|---|
| K18-R: | 5′-TCCACTTCCACAGTCAATCCA-3′, |

(SEQ ID NO: 27)

| | |
|---|---|
| K14-F: | 5′-AGCGGCAAGAGTGAGATTTCT-3′, |

(SEQ ID NO: 28)

| | |
|---|---|
| K14-R: | 5′-CCTCCAGGTTATTCTCCAGGG-3′, |

(SEQ ID NO: 29)

| | |
|---|---|
| K5-F: | 5′-GAGATCGCCACCTACAGGAA-3′, |

(SEQ ID NO: 30)

| | |
|---|---|
| K5-R: | 5′-TCCTCCGTAGCCAGAAGAGA-3′, |

(SEQ ID NO: 31)

| | |
|---|---|
| Vimentin-F: | 5′-CGGCTGCGAGAGAAATTGC-3′, |

(SEQ ID NO: 32)

| | |
|---|---|
| Vimentin-R: | 5′-CCACTTTCCGTTCAAGGTCAAG-3′, |

(SEQ ID NO: 33)

| | |
|---|---|
| β-Actin-F: | 5′-CGGTTCCGATGCCCTGAGGCTCTT-3′, |

(SEQ ID NO: 34)

| | |
|---|---|
| β-Actin-R: | 5′-CGTCACACTTCATGATGGAATTGA-3′. |

1 Levy-Lahad, E. & Friedman, E. Cancer risks among BRCA1 and BRCA2 mutation carriers. Br J Cancer 96, 11-15 (2007).
2 Lim, E. et al. Aberrant luminal progenitors as the candidate target population for basal tumor development in BRCA1 mutation carriers. Nat Med 15, 907-913 (2009).
3 Molyneux, G. et al. BRCA1 basal-like breast cancers originate from luminal epithelial progenitors and not from basal stem cells. Cell Stem Cell 7, 403-417 (2010).
4 Proia, T. A. et al. Genetic predisposition directs breast cancer phenotype by dictating progenitor cell fate. Cell Stem Cell 8, 149-163 (2011).
5 Visvader, J. E. Keeping abreast of the mammary epithelial hierarchy and breast tumorigenesis. Genes Dev 23, 2563-2577 (2009).
6 Rios, A. C., Fu, N. Y., Lindeman, G. J. & Visvader, J. E. In situ identification of bipotent stem cells in the mammary gland. Nature 506, 322-327 (2014).
7 Marquis, S. T. et al. The developmental pattern of Brca1 expression implies a role in differentiation of the breast and other tissues. Nat Genet. 11, 17-26 (1995).
8 King, M. C. “The race” to clone BRCA1. Science 343, 1462-1465 (2014).
9 Silver, D. P. & Livingston, D. M. Mechanisms of BRCA1 tumor suppression. Cancer Discov 2, 679-684 (2012).
10 Venkitaraman, A. R. Cancer Suppression by the Chromosome Custodians, BRCA1 and BRCA2. Science 343, 1470-1475 (2014).
11 Lee, E. Y. & Abbondante, S. Tissue-specific tumor suppression by BRCA1. Proc Natl Acad Sci USA 111, 4353-4354 (2014).
12 Couch, F. J., Nathanson, K. L. & Offit, K. Two decades after BRCA: setting paradigms in personalized cancer care and prevention. Science 343, 1466-1470 (2014).
13 Xu, X. L. et al. Rb suppresses human cone-precursor-derived retinoblastoma tumours. Nature 514, 385-388 (2014).
14 Bremner, R. & Sage, J. Cancer: The origin of human retinoblastoma. Nature 514, 312-313 (2014).
15 Derheimer, F. A. & Kastan, M. B. Multiple roles of ATM in monitoring and maintaining DNA integrity. FEBS Lett 584, 3675-3681 (2010).
16 Valentin-Vega, Y. A. et al. Mitochondrial dysfunction in ataxia-telangiectasia. Blood 119, 1490-1500 (2012).
17 Huen, M. S., Sy, S. M. & Chen, J. BRCA1 and its toolbox for the maintenance of genome integrity. Nat Rev Mol Cell Biol 11, 138-148 (2010).
18 Moynahan, M. E. & Jasin, M. Mitotic homologous recombination maintains genomic stability and suppresses tumorigenesis. Nat Rev Mol Cell Biol 11, 196-207 (2010).
19 Zhang, J. & Powell, S. N. The role of the BRCA1 tumor suppressor in DNA double-strand break repair. Mol Cancer Res 3, 531-539 (2005).
20 Xu, X. et al. Genetic interactions between tumor suppressors BRCA1 and p53 in apoptosis, cell cycle and tumorigenesis. Nat Genet 28, 266-271 (2001).
21 Parameswaran, B. et al. Damage-Induced BRCA1 Phosphorylation by Chk2 Contributes to the Timing of End Resection. Cell Cycle 4, 437-448 (2015).
22 Zhu, Q. et al. BRCA1 tumour suppression occurs via heterochromatin-mediated silencing. Nature 477, 179-184 (2011).
23 Rosen, E. M., Fan, S. & Ma, Y. BRCA1 regulation of transcription. Cancer Lett 236, 175-185 (2006).
24 Scully, R. et al. BRCA1 is a component of the RNA polymerase II holoenzyme. Proc Natl Acad Sci USA 94, 5605-5610 (1997).
25 Fan, S. et al. BRCA1 inhibition of estrogen receptor signaling in transfected cells. Science 284, 1354-1356 (1999).
26 Tkocz, D. et al. BRCA1 and GATA3 corepress FOXC1 to inhibit the pathogenesis of basal-like breast cancers. Oncogene 31, 3667-3678 (2012).
27 Gorski, J. J. et al. Profiling of the BRCA1 transcriptome through microarray and ChIP-chip analysis. Nucleic Acids Res 39, 9536-9548 (2011).
28 Consortium, E. P. et al. An integrated encyclopedia of DNA elements in the human genome. Nature 489, 57-74 (2012).
29 Gardini, A., Baillat, D., Cesaroni, M. & Shiekhattar, R. Genome-wide analysis reveals a role for BRCA1 and PALB2 in transcriptional co-activation. EMBO J 33, 890-90 (2014).
30 Bhatia, V. et al. BRCA2 prevents R-loop accumulation and associates with TREX-2 mRNA export factor PCID2. Nature 511, 362-365 (2014).
31 Hill, S. J. et al. Systematic screening reveals a role for BRCA1 in the response to transcription-associated DNA damage. Genes Dev 28, 1957-1975 (2014).
32 Hatchi, E. et al. BRCA1 Recruitment to Transcriptional Pause Sites Is Required for R-Loop-Driven DNA Damage Repair. Mol Cell 57, 636-647 (2015).
33 Svejstrup, J. Q. The interface between transcription and mechanisms maintaining genome integrity. Trends Biochem Sci 35, 333-338 (2010).
34 Gan, W. et al. R-loop-mediated genomic instability is caused by impairment of replication fork progression. Genes Dev 25, 2041-2056 (2011).
35 Kim, N. & Jinks-Robertson, S. Transcription as a source of genome instability. Nat Rev Genet 13, 204-214 (2012).
36 Saponaro, M. et al. RECQLS controls transcript elongation and suppresses genome instability associated with transcription stress. Cell 157, 1037-1049 (2014).
37 Aguilera, A. & Garcia-Muse, T. R loops: from transcription byproducts to threats to genome stability. Mol Cell 46, 115-124 (2012).
38 Skourti-Stathaki, K. & Proudfoot, N. J. A double-edged sword: R loops as threats to genome integrity and powerful regulators of gene expression. Genes Dev 28, 1384-1396 (2014).
39 Hamperl, S. & Cimprich, K. A. The contribution of co-transcriptional RNA:DNA hybrid structures to DNA damage and genome instability. DNA Repair (Amst) 19, 84-94 (2014).
40 Groh, M. & Gromak, N. Out of balance: R-loops in human disease. PLoS Genet 10, e1004630 (2014).
41 Aguilera, A. The connection between transcription and genomic instability. EMBO J 21, 195-201 (2002).
42 Huertas, P. & Aguilera, A. Cotranscriptionally formed DNA:RNA hybrids mediate transcription elongation impairment and transcription-associated recombination. Mol Cell 12, 711-721 (2003).
43 Skourti-Stathaki, K., Proudfoot, N. J. & Gromak, N. Human senataxin resolves RNA/DNA hybrids formed at transcriptional pause sites to promote Xrn2-dependent termination. Mol Cell 42, 794-805 (2011).
44 Skourti-Stathaki, K., Kamieniarz-Gdula, K. & Proudfoot, N. J. R-loops induce repressive chromatin marks over mammalian gene terminators. Nature 516, 436-439 (2014).
45 Powell, W. T. et al. R-loop formation at Snord116 mediates topotecan inhibition of Ube3a-antisense and allele-specific chromatin decondensation. Proc Natl Acad Sci USA 110, 13938-13943 (2013).
46 Sun, Q., Csorba, T., Skourti-Stathaki, K., Proudfoot, N. J. & Dean, C. R-loop stabilization represses antisense transcription at the Arabidopsis FLC locus. Science 340, 619-621 (2013).
47 Ginno, P. A., Lott, P. L., Christensen, H. C., Korf, I. & Chedin, F. R-loop formation is a distinctive characteristic of unmethylated human CpG island promoters. Mol Cell 45, 814-825 (2012).
48 Nakama, M., Kawakami, K., Kajitani, T., Urano, T. & Murakami, Y. DNA-RNA hybrid formation mediates RNAi-directed heterochromatin formation. Genes Cells 17, 218-233 (2012).
49 Ye, Q. et al. BRCA1-induced large-scale chromatin unfolding and allele-specific effects of cancer-predisposing mutations. J Cell Biol 155, 911-921 (2001).
50 Yamaguchi, Y. et al. NELF, a multisubunit complex containing RD, cooperates with DSIF to repress RNA polymerase II elongation. Cell 97, 41-51 (1999).
51 Narita, T. et al. Human transcription elongation factor NELF: identification of novel subunits and reconstitution of the functionally active complex. Mol Cell Biol 23, 1863-1873 (2003).
52 Levine, M. Paused RNA Polymerase II as a Developmental Checkpoint. Cell 145, 502-511 (2011).
53 Kwak, H. & Lis, J. T. Control of Transcriptional Elongation. Annu Rev Genet 47, 483-508 (2013).
54 Adelman, K. & Lis, J. T. Promoter-proximal pausing of RNA polymerase II: emerging roles in metazoans. Nat Rev Genet 13, 720-731 (2012).
55 Gilchrist, D. A. et al. Pausing of RNA polymerase II disrupts DNA-specified nucleosome organization to enable precise gene regulation. Cell 143, 540-551 (2010).
56 Sun, J. & Li, R. Human negative elongation factor activates transcription and regulates alternative transcription initiation. J Biol Chem 285, 6443-6452 (2010).
57 Aiyar, S. E. et al. Attenuation of estrogen receptor alpha-mediated transcription through estrogen-stimulated recruitment of a negative elongation factor. Genes Dev 18, 2134-2146 (2004).
58 Kininis, M., Isaacs, G. D., Core, L. J., Hah, N. & Kraus, W. L. Postrecruitment regulation of RNA polymerase II directs rapid signaling responses at the promoters of estrogen target genes. Mol Cell Biol 29, 1123-1133 (2009).
59 Danko, C. G. et al. Signaling pathways differentially affect RNA polymerase II initiation, pausing, and elongation rate in cells. Mol Cell 50, 212-222 (2013).
60 Jennings, B. H. Pausing for thought: disrupting the early transcription elongation checkpoint leads to developmental defects and tumourigenesis. Bioessays 35, 553-560 (2013).
61 Amleh, A. et al. Mouse Cofactor of BRCA1 (Cobral) is Required for Early Embryogenesis. PloS One 4, e5034 (2009).
62 Williams, L. H. et al. Pausing of RNA Polymerase II Regulates Mammalian Developmental Potential through Control of Signaling Networks. Mol Cell (2015).
63 Pan, H. et al. RNA Polymerase II Pausing Factor NELF Controls Energy Homeostasis in Cardiomyocytes. Cell Rep 7, 79-85 (2014).
64 Wagner, K. U. et al. Cre-mediated gene deletion in the mammary gland. Nucleic Acids Res 27, 4323-4330 (1997).
65 Shackleton, M. et al. Generation of a functional mammary gland from a single stem cell. Nature 439, 84-88 (2006).
66 Xu, X. et al. Conditional mutation of Brca1 in mammary epithelial cells results in blunted ductal morphogenesis and tumour formation. Nat Genet 22, 37-43 (1999).
67 Smart, C. E. et al. Analysis of Brca1-deficient mouse mammary glands reveals reciprocal regulation of Brca1 and c-kit. Oncogene 30, 1597-1607 (2011).
68 Kim, W. Y. & Sharpless, N. E. The regulation of INK4/ARF in cancer and aging. Cell 127, 265-275 (2006).
69 Collado, M., Blasco, M. A. & Serrano, M. Cellular senescence in cancer and aging. Cell 130, 223-233 (2007).
70 Jacobs, J. J., Kieboom, K., Marino, S., DePinho, R. A. & van Lohuizen, M. The oncogene and Polycomb-group gene bmi-1 regulates cell proliferation and senescence through the ink4a locus. Nature 397, 164-168 (1999).
71 Pietersen, A. M. et al. Bmi1 regulates stem cells and proliferation and differentiation of committed cells in mammary epithelium. Curr Biol 18, 1094-1099 (2008).
72 Bruggeman, S. W. et al. Ink4a and Arf differentially affect cell proliferation and neural stem cell self-renewal in Bmi1-deficient mice. Genes Dev 19, 1438-1443 (2005).
73 Molofsky, A. V., He, S., Bydon, M., Morrison, S. J. & Pardal, R. Bmi-1 promotes neural stem cell self-renewal and neural development but not mouse growth and survival by repressing the p16Ink4a and p19Arf senescence pathways. Genes Dev 19, 1432-1437 (2005).
74 Biehs, B. et al. BMI1 represses Ink4a/Arf and Hox genes to regulate stem cells in the rodent incisor. Nat Cell Biol 15, 846-852 (2013).
75 Hakem, R. et al. The Tumor Suppressor Gene Brcal Is Required for Embryonic Cellular Proliferation in the Mouse. Cell 85, 1009-1023 (1996).
76 Hakem, R., de la Pompa, J. L., Elia, A., Potter, J. & Mak, T. W. Partial rescue of Brca1 (5-6) early embryonic lethality by p53 or p21 null mutation. Nat Genet 16, 298-302 (1997).
77 Ludwig, T., Chapman, D. L., Papaioannou, V. E. & Efstratiadis, A. Targeted mutations of breast cancer susceptibility gene homologs in mice: lethal phenotypes of Brca1, Brca2, Brca1/Brca2, Brca1/p53, and Brca2/p53 nullizygous embryos. Genes Dev 11, 1226-1241 (1997).
78 McBryan, J., Howlin, J., Kenny, P. A., Shioda, T. & Martin, F. ERalpha-CITED1 co-regulated genes expressed during pubertal mammary gland development: implications for breast cancer prognosis. Oncogene 26, 6406-6419 (2007).
79 Lu, S. et al. Transcriptional responses to estrogen and progesterone in mammary gland identify networks regulating p53 activity. Endocrinology 149, 4809-4820 (2008).
80 Deng, C. X. & Xu, X. Generation and analysis of Brcal conditional knockout mice. Methods Mol Biol 280, 185-200 (2004).
81 Shehata, M. et al. Phenotypic and functional characterization of the luminal cell hierarchy of the mammary gland. Breast Cancer Res 14, R134 (2012).
82 Cao, L. et al. A selective requirement for 53BP1 in the biological response to genomic instability induced by Brcal deficiency. Mol Cell 35, 534-541 (2009).
83 Bunting, S. F. et al. 53BP1 inhibits homologous recombination in Brca1-deficient cells by blocking resection of DNA breaks. Cell 141, 243-254 (2010).
84 Pierce, A. J., Hu, P., Han, M., Ellis, N. & Jasin, M. Ku DNA end-binding protein modulates homologous repair of double-strand breaks in mammalian cells. Genes Dev 15, 3237-3242 (2001).
85 Ira, G. et al. DNA end resection, homologous recombination and DNA damage checkpoint activation require CDK1. Nature 431, 1011-1017 (2004).
86 Jazayeri, A. et al. ATM- and cell cycle-dependent regulation of ATR in response to DNA double-strand breaks. Nat Cell Biol 8, 37-45 (2006).
87 Scully, R. et al. Association of BRCA1 with Rad51 in mitotic and meiotic cells. Cell 88, 265-275 (1997).
88 Phillips, D. D. et al. The sub-nanomolar binding of DNA-RNA hybrids by the single-chain FAT fragment of antibody S9.6. J Mol Recognit 26, 376-381 (2013).
89 Hu, Y.-F. et al. Modulation of aromatase expression by BRCA1: a possible link to tissue-specific tumor suppression. Oncogene 24, 8343-8348 (2005).
90 Poole, A. J. et al. Prevention of Brcal-mediated mammary tumorigenesis in mice by a progesterone antagonist. Science 314, 1467-1470 (2006).
91 Gorrini, C. et al. Estrogen controls the survival of BRCA1-deficient cells via a PI3K-NRF2-regulated pathway. Proc Natl Acad Sci USA 111, 4472-4477 (2014).
92 Tomasetti, C. & Vogelstein, B. Cancer etiology. Variation in cancer risk among tissues can be explained by the number of stem cell divisions. Science 347, 78-81 (2015).
93 Rosen, J. M. & Roarty, K. Paracrine signaling in mammary gland development: what can we learn about intratumoral heterogeneity? Breast Cancer Res 16, 202 (2014).
94 Visvader, J. E. & Stingl, J. Mammary stem cells and the differentiation hierarchy: current status and perspectives. Genes Dev 28, 1143-1158 (2014).
95 Radovich, M. et al. Characterizing the heterogeneity of triple-negative breast cancers using microdissected normal ductal epithelium and RNA-sequencing. Breast Cancer Res Treat 143, 57-68 (2014).
96 Gyorffy, B. et al. An online survival analysis tool to rapidly assess the effect of 22,277 genes on breast cancer prognosis using microarray data of 1,809 patients. Breast Cancer Res Treat 123, 725-731 (2010).
97 Stingl, J. et al. Purification and unique properties of mammary epithelial stem cells. Nature. 439, 993-997 (2006).
98 Ransburgh, D. J., Chiba, N., Ishioka, C., Toland, A. E. & Parvin, J. D. Identification of breast tumor mutations in BRCA1 that abolish its function in homologous DNA recombination. Cancer Res 70, 988-995 (2010).
99 Li, H. & Durbin, R. Fast and accurate short read alignment with Burrows-Wheeler transform. Bioinformatics 25, 1754-1760 (2009).
100 Lan, X., Bonneville, R., Apostolos, J., Wu, W. & Jin, V. X. W-ChIPeaks: a comprehensive web application tool for processing ChIP-chip and ChIP-seq data. Bioinformatics 27, 428-430 (2011).
101 Hulsen, T., de Vlieg, J. & Alkema, W. BioVenn—a web application for the comparison and visualization of biological lists using area-proportional Venn diagrams. BMC Genomics 9, 488 (2008).

We claim:

1. A method of diagnosing whether a subject is at risk of developing breast cancer, comprising
(a) obtaining a biological sample from the subject;
(b) measuring the level of R-loop in the biological sample by conducting at least one hybridization assay of the biological sample so as to obtain physical data to determine whether the level of R-loop in the biological sample is higher than the level of R-loop in a control sample from a non-BRCA mutation carrier;
(c) comparing the level of R-loop in the biological sample with the level of R-loop in a control sample from a non-BRCA mutation carrier; and
(d) identifying the subject is at risk of developing breast cancer if the physical data indicate that the level of R-loop in the biological sample is higher than the level of R-loop in a control sample from a non-BRCA mutation carrier.

2. The method of claim 1, wherein the subject is a BRCA mutation carrier.

3. The method of claim 2, wherein the BRCA mutation carrier is a BRCA1 or BRCA2 mutation carrier.

4. The method of claim 1, wherein the non-BRCA mutation carrier is a subject having two wild type copies of the BRCA gene.

5. The method of claim 1, wherein the hybridization assay includes an ELISPOT assay, ELISA, fluorescent immunoassays, two-antibody sandwich assays, a flow-through or strip test format, PCR, Real time PCR, Reverse Transcription-PCR (RT-PCR), immunohistochemistry, or DNA/RNA immunoprecipitation.

6. The method of claim 1, the hybridization assay is carried out with an R-loop antibody.

7. The method of claim 1, wherein the sample comprises one or more of tissue, blood, bone marrow, plasma, serum, urine, and feces.

8. The method of claim 7, wherein the sample comprises breast tissue.

9. The method of claim 7, wherein the sample comprises epithelial cells.

10. The method of claim 9, wherein epithelial cells are luminal epithelial cells.

11. The method of claim 1, further comprising administering to said subject a therapeutically effective amount of a given therapeutic.

12. A method for treating breast cancer in a subject, comprising administering to said subject a therapeutically effective amount of a given therapeutic when the subject is diagnosed with increased risk of developing breast cancer by the steps of
(a) obtaining a biological sample from the subject;
(b) measuring the level of R-loop in the biological sample by conducting at least one hybridization assay of the biological sample so as to obtain physical data to determine whether the level of R-loop in the biological sample is higher than the level of R-loop in a control sample from a non-BRCA mutation carrier;
(c) comparing the level of R-loop in the biological sample with the level of R-loop in a control sample from a non-BRCA mutation carrier; and
(d) identifying the subject is at risk of developing breast cancer if the physical data indicate that the level of R-loop in the biological sample is higher than the level of R-loop in a control sample from a non-BRCA mutation carrier.

13. A method of treating a subject having an increase in breast epithelium R-loop comprising administering a treatment to the subject that reduces or eliminates R-loop, wherein the subject is a BRCA1 mutation carrier.

14. The method of claim 13, wherein the treatment is increasing expression and/or activity of RNase H or decreasing COBRA1 expression and/or activity.

15. A method of reducing tumor incidence in BRCA1-deficient subjects comprising administering a treatment that reduces or eliminates Cobra1 activity.

16. The method of claim 15, wherein the treatment comprises siRNA that targets Cobra1 mRNA.

17. A method of increasing mammary gland development in Cobra-deficient subjects comprising administering a treatment that reduces or eliminates BRCA1 activity.

18. The method of claim 17, wherein the treatment comprises siRNA.

19. The method of claim 17, wherein the siRNA targets BRCA1 mRNA.

20. A method of increasing mammary gland development in Cobra-deficient subjects comprising administering a treatment that alters transcription of one or more puberty-related genes, estrogen-responsive genes or progesterone-responsive genes.

21. The method of claim 20, wherein the puberty-related genes are Gata3, Prlr, Ramp2, Vwf, Prom2, or Acot1.

22. The method of claim 20, wherein the estrogen-responsive genes are 2410081M15RIK, 6430706D22RIK, ACOT1, ACTB, ARL4A, BCL6B, CTSH, CXCL9, EMCN, GBP6, GGTA1, HOXA7, PABPC1, PDLIM1, PDLIM2, PROM2, PTPN14, SLCO2B1, STARD10, TMEM2, WIPI1, or a combination thereof.

23. The method of claim 20, wherein the progesterone-responsive genes are 5730593F17RIK, CCDC80, CDH13, CLDN5, CRYZ, EDN1, IRX1, NOXO1, PKP2, PRKCDBP, PSEN2, SLC7A3, SPNB3, or a combination thereof.

24. The method of claim 20, wherein a treatment that alters transcription comprises a treatment that increases transcription.

25. The method of claim 20, wherein a treatment that alters transcription comprises a treatment that decreases transcription.
